(19) United States
(12) Patent Application Publication
(10) Pub. No.: US 2026/0185144 A1
(43) Pub. Date: Jul. 2, 2026

(54) ORTHOGONAL CROSSLINKING POLYMER SYSTEMS FOR IN SITU ANALYTE DETECTION

(71) Applicant: 10x Genomics, Inc., Pleasanton, CA (US)

(72) Inventors: Cheyenne CHRISTOPHERSON, San Ramon, CA (US); Shalini GOHIL, Welleseley, MA (US); Cheyenne LIU, Dublin, CA (US); Minkyung PARK, Dublin, CA (US); Meiliana TJANDRA, Dublin, CA (US)

(21) Appl. No.: 19/070,455

(22) Filed: Mar. 4, 2025

Related U.S. Application Data
(60) Provisional application No. 63/561,686, filed on Mar. 5, 2024.

(51) Int. Cl. C12Q 1/6841; C12Q 1/6806; C12Q 1/682

(52) U.S. Cl. CPC C12Q 1/6841; C12Q 1/6806; C12Q 1/682

(57) ABSTRACT

The present disclosure relates in some aspects to methods and compositions for multi-functional matrix formation. In some embodiments, the multi-functional matrix is formed using a multi-arm monomer or polymer comprising a plurality of arms converging at a central branching point, each arm comprising a functional group R^{A}, and (ii) a second monomer or polymer comprising at least two functional groups R^{B} and a pendant tethering moiety R^{T}.

### CROSS-REFERENCE TO RELATED APPLICATIONS

The application claims priority to U.S. Provisional Patent Application No. 63/561,686, filed Mar. 5, 2024, which is herein incorporated by reference in its entirety.

### FIELD

The present disclosure relates in some aspects to methods for processing biological samples comprising formation of a multi-functional matrix.

### BACKGROUND

Despite improvements in transcriptomic analysis, many nucleic acid analytes present in biological samples can be lost throughout sample preparation using standard protocols and reagents, e.g., permeabilization and de-crosslinking, to enable analysis and imaging, such as by fluorescence in situ hybridization. Although the extent of losses of these uncaptured nucleic acid analytes out of the RNA transcripts for a given sample remain unknown, the undetected RNA analytes nonetheless represent substantial segment of the overall transcriptome that remains omitted by analysis. Existing methods for embedding biological samples in matrices for analysis suffer from challenges related to the positional stability of analytes, probes, or amplification products.

Thus, improved methods and techniques for processing and analyzing biological samples to detect analytes of interest are needed. Provided herein are methods and compositions that address such and other needs.

### SUMMARY

In some aspects, provided herein are novel methods, compositions, and kits for matrix-embedding of biological samples. The provided methods, compositions, and kits in some aspects address challenges of whole transcriptome decoding by sequential probe hybridization or by in situ sequencing approaches by allowing stable anchoring of analytes or other products or labelling agents (e.g., rolling circle amplification products or labelling agents such as antibodies) to a matrix. In some aspects, the improved matrix embedding methods, compositions, and kits allow for improved tissue clearing to remove autofluorescence sources from a biological sample, improving the ability to detect target analytes above background. In some aspects, the provided methods, compositions, systems and kits reduce target analyte mis-localization by providing improved anchoring of analytes or other products or labelling agents.

In some embodiments, the matrix embedding methods and compositions include use of orthogonal chemistries for matrix formation and anchoring of analytes or associated probes or products thereof. In certain aspects, the provided methods, compositions, and kits provide greater matrix network uniformity together with allowing covalent anchoring of molecules such as amplification products to the matrix using orthogonal chemistries. The present application thus provides substantial improvements over existing methods and gel networks, which are random networks that can suffer from inhomogeneity and that do not include orthogonal chemistry to crosslink RCPs. Provided herein is a method for processing a biological sample, the method comprising contacting the biological sample with at least a multi-arm monomer or polymer comprising a plurality of arms converging at a central branching point, each arm comprising a functional group R^{A}, and a second monomer or polymer comprising at least two functional groups R^{B} and a pendant tethering moiety R^{T}.

In one aspect, provided herein is a method for processing a biological sample, the method comprising:
a) contacting the biological sample with: (i) a multi-arm monomer or polymer comprising a plurality of arms converging at a central branching point, each arm comprising a functional group R^{A}, and (ii) a second monomer or polymer comprising at least two functional groups R^{B} and a pendant tethering moiety R^{T};
b) forming a matrix embedding the biological sample, wherein the matrix is the product of a polymerization reaction between the R^{A} of the multi-arm monomer or polymer and R^{B} of the second monomer or polymer; and
c) attaching R^{T} to an attachment moiety that is directly or indirectly attached to an analyte or labeling agent associated with an analyte, or a nucleic acid amplification product in the biological sample, or any combination thereof;
thereby attaching the analyte, labeling agent, or nucleic acid amplification product, or any combination thereof to the matrix.

In some embodiments, the second monomer or polymer is a monomer or polymer with two functional groups and without a central branching point. In some embodiments, the second monomer or polymer comprises a plurality of arms converging at a central branching point, each arm comprising a R^{B}, optionally wherein the second monomer or polymer has four arms converging at a central branching point. In some embodiments, the second monomer or polymer comprises a poly(ethylene glycol). In some embodiments, the second monomer or polymer comprises a polyacrylamide. In some embodiments, the second monomer or polymer comprises a peptide. In some embodiments, the peptide is between about 5 and about 20 amino acid residues in length. In some embodiments, both the N-terminal and C-terminal amino acid residues of the peptide comprise the R^{B}. In some embodiments, the peptide comprises cysteine or lysine. In some embodiments, the pendant tethering moiety R^{T} is attached to an amino acid residue between two R^{B}. In some embodiments, the second monomer or polymer comprises at least two pendant tethering moieties R^{T}, each of which is independently attached to an independently selected amino acid residue between two R^{B}. In some embodiments, the multi-arm monomer or polymer is a 3-arm monomer or polymer. In some embodiments, the multi-arm monomer or polymer is a 4-arm monomer or polymer. In some embodiments, R^{T} is substantially unreacted when R^{A} reacts with R^{B} to form the matrix in b). In some embodiments, R^{T} does not react with R^{A} or R^{B} under conditions used for forming the matrix in b). In some embodiments, the polymerization reaction and the attaching of R^{T} to the attachment moiety are performed using orthogonal chemical reactions. In some embodiments, the contacting in a) comprises contacting the biological sample with a matrix-forming composition comprising the multi-arm monomer or polymer and the second monomer or polymer. In some embodiments, the contacting in a) comprises contacting the biological sample with the first monomer or polymer and the second monomer or polymer sequentially, in either order. In some embodiments, the forming the matrix in b) and the attaching the tethering moiety to the cognate attachment moiety in c) are performed simultaneously. In some embodiments, attaching the tethering moiety to the cognate attachment moiety in c) is performed after forming the matrix in b).

In some embodiments, the method further comprises clearing the biological sample.

In some embodiments, the multi-arm monomer or polymer is a compound according to Formula I:

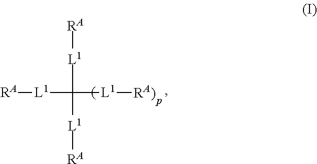

or a salt thereof,
wherein p is 0 or 1;
wherein L¹ is an C₅-C₁₀₀ alkylene optionally interrupted by one or more groups independent selected from the group consisting of —NH—, —O—, —S—, —SO₂—, —N(C₁₋₆ alkyl)-, C₆-C₁₀ aryl, 5- to 6-membered heteroaryl, C₃-C₈ cycloalkyl, and 5- to 6-membered heterocycle, and L¹ is optionally substituted with one or more substituents independently selected from the group consisting of oxo, halo, —OH, —CN, C₁-C₆ alkoxy, C₁-C₆ haloalkoxy, or a side chain of an amino acid;
and
the second monomer or polymer is a compound according to Formula II:

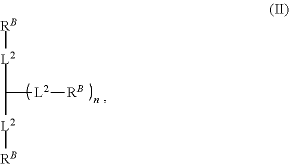

or a salt thereof,
wherein n is 0, 1, or 2;
wherein L² is an C₅-C₁₀₀ alkylene optionally interrupted by one or more groups independent selected from the group consisting of —NH—, —O—, —S—, —SO₂—, —N(C₁₋₆ alkyl)-, a C₆-C₁₀ aryl, 5- to 6-membered heteroaryl, C₃-C₈ cycloalkyl, and 5- to 6-membered heterocycle, and L² is optionally substituted with one or more substituents independently selected from the group consisting of oxo, halo, —OH, —CN, amide, C₁-C₆ alkoxy, C₁-C₆ haloalkoxy, and a side chain of an amino acid;
wherein L² is further attached to one or more tethering moiety R^{T};
wherein R^{A} is capable of reacting with R^{B} to form the matrix of b); and
wherein R^{T} is substantially unreacted when R^{A} reacts with R^{B} to form the matrix in b).

In some embodiments, R^{A} is a nucleophilic group and R^{B} is an electrophilic group; or R^{A} is an electrophilic group and R^{B} is a nucleophilic group; or R^{A} and R^{B} are a click chemistry pair. In some embodiments, L¹ comprises a PEG unit. In some embodiments, L¹ comprises a polyacrylamide unit. In some embodiments, L² comprises a PEG unit. In some embodiments, L² comprises a polyacrylamide unit. In some embodiments, L² comprises a peptide. In some embodiments, R^{A} is a thiol group and R^{B} is a maleimide group, or wherein R^{A} is a maleimide group and R^{B} is a thiol group. In some embodiments, the polymerization reaction is a thiol-maleimide Michael coupling reaction. In some embodiments, n is 0, and R^{B} comprises —SH. In some embodiments, R^{T} comprises phenol, amide, or a combination thereof.

In some embodiments, the second monomer or polymer is of Formula (II-1)

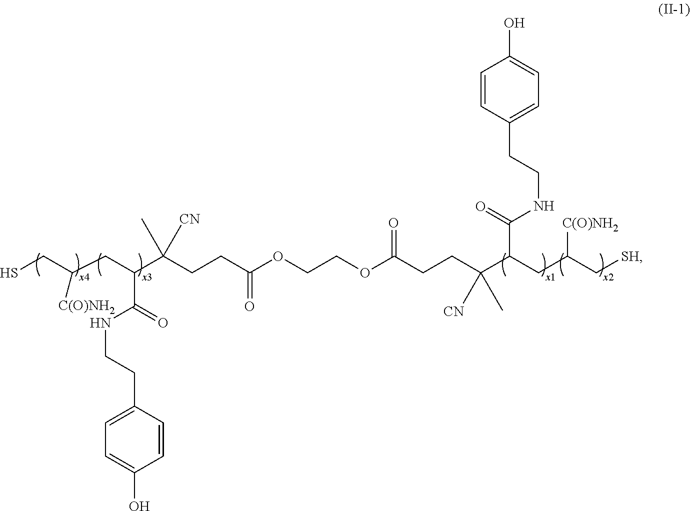

wherein ×1, ×2, ×3, and ×4 are each independently an integer from 1 to 10.

In some embodiments, n is 2 and R^{B} comprises-SH.

In some embodiments, the second monomer or polymer is of Formula (II-2):

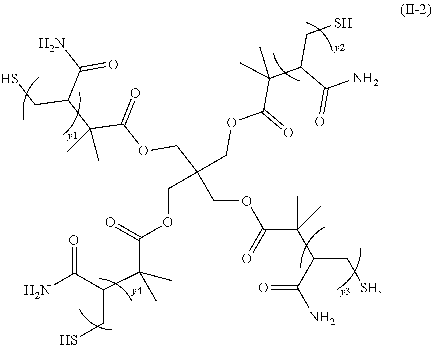

wherein y1, y2, y3, and y4 are each independently an integer from 1 to 10.

In some embodiments, R^{A} comprises

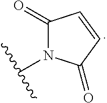 [SMILES: *N1C(=O)C=CC1=O]

In some embodiments, the multi-arm monomer or polymer is of Formula (I-1)

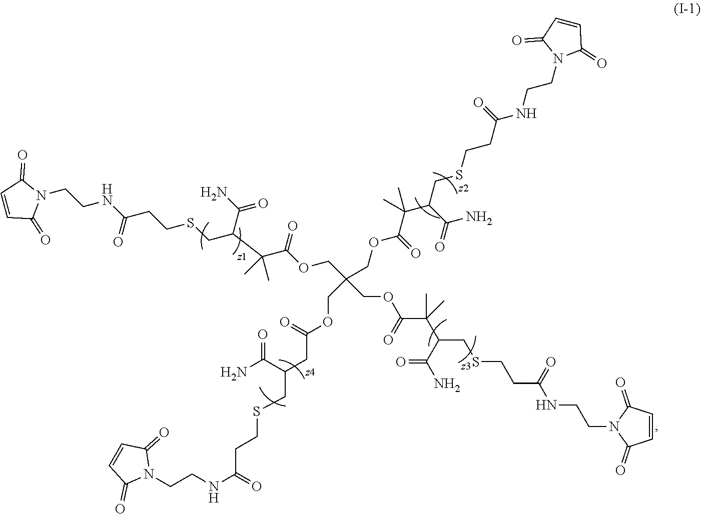

wherein z1, z2, z3, and z4 are each independently an integer from 1 to 10.

In some embodiments, R^{A} is an azide group and R^{B} is an alkyne group, or wherein R^{A} is an alkyne group and R^{B} is an azide group. In some embodiments, the polymerization reaction is a copper-ion-catalyzed azide-alkyne cycloaddition reaction (CuAAC reaction). In some embodiments, the biological sample is on an alkyne-modified substrate.

In some embodiments, R^{A} is a N-hydroxysuccinimide ester (NHS) group and R^{B} is a primary amine group, or wherein R^{A} is a primary amine group and R^{B} is a NHS group. In some embodiments, the polymerization reaction is performed at a pH between about 7 and about 9.

In some embodiments, R^{T} comprises a phenol group and the attachment moiety comprises an amine group, or R^{T} comprises an amine group and the attachment moiety comprises a phenol group, optionally wherein attaching R^{T} to the attachment moiety comprises performing a tyrosinase reaction. In some embodiments, R^{T} comprises an alkene group and the attachment moiety comprises a thiol group, or R^{T} comprises a thiol group and the attachment moiety comprises an alkene group, optionally wherein attaching R^{T} to the attachment moiety comprises performing a thiol-ene addition in the presence of ultraviolet irradiation. In some embodiments, R^{T} comprises an amide group and the attachment moiety comprises an amine group, or R^{T} comprises an amine group and the attachment moiety comprises an amide group, optionally wherein attaching R^{T} to the attachment moiety comprises performing amide-amine coupling with transglutaminase. In some embodiments, R^{T} comprises an azide group and the attachment moiety comprises an alkyne group, or R^{T} comprises an alkyne group and the attachment moiety comprises an azide group, optionally wherein attaching R^{T} to the attachment moiety comprises performing a copper-ion-catalyzed azide-alkyne cycloaddition reaction (CuAAC reaction).

In some embodiments, the biological sample is on a chemically modified substrate. In some embodiments, the chemically modified substrate is capable of immobilizing the multi-arm monomer or polymer, the second monomer or polymer, or both. In some embodiments, the chemically modified substrate is capable of forming covalent bond with R^{A} of the multi-arm monomer or polymer, R^{B} of the second monomer or polymer, or R^{T} of the second monomer or polymer. In some embodiments, the chemically modified substrate comprises an NHS ester, and at least one of R^{A}, R^{B}, or R^{T} comprises —NH₂. In some embodiments, the chemically modified substrate comprises an acrylamide, and at least one of R^{A}, R^{B}, or R^{T} comprises an acrylamide. In some embodiments, the chemically modified substrate is capable of forming covalent bond with a linking reagent, and the linking reagent is capable of forming covalent bond with R^{A} of the multi-arm monomer or polymer, R^{B} of the second monomer or polymer, or R^{T} of the second monomer or polymer. In some embodiments, the chemically modified substrate comprises an NHS ester, and wherein the linking reagent comprises —NH₂ and a moiety capable of forming covalent bond with R^{A} of the multi-arm monomer or polymer, R^{B} of the second monomer or polymer, or R^{T} of the second monomer or polymer. In some embodiments, the chemically modified substrate comprises an acrylamide, and wherein the linking reagent comprises an acrylamide and a moiety capable of forming covalent bond with R^{A} of the multi-arm monomer or polymer, R^{B} of the second monomer or polymer, or R^{T} of the second monomer or polymer.

In some embodiments, the analyte is a nucleic acid analyte, optionally wherein the analyte is an RNA present in the biological sample. In some embodiments, the method comprises contacting the biological sample or the matrix with a nucleic acid probe or probe set that hybridizes to a target sequence in the nucleic acid analyte. In some embodiments, the attachment agent is directly or indirectly attached to a labeling agent that binds to the nucleic acid analyte, and wherein the labeling agent is the nucleic acid probe or probe set. In some embodiments, the nucleic acid probe or probe set is (i) a circular probe, or (ii) a circularizable probe or probe set, wherein the method comprises circularizing the circularizable probe or probe set to form a circularized probe. In some embodiments, the method comprises generating a rolling circle amplification (RCA) product from the circular or circularized probe in the biological sample or matrix. In some embodiments, the attachment moiety is directly or indirectly attached to a nucleic acid amplification product in the biological sample, and wherein the nucleic acid amplification product is the RCA product. In some embodiments, the attachment moiety is in a primer used to form the RCA product, and wherein the primer comprises a reverse complement of a primer binding sequence in the circular or circularized probe. In some embodiments, the attachment moiety is in a modified nucleotide that is incorporated into the RCA product.

In some embodiments, the analyte is a non-nucleic acid analyte. In some embodiments, the attachment agent is directly or indirectly attached to a labeling agent that binds to the analyte, optionally wherein the method comprises contacting the biological sample or the matrix with the labeling agent. In some embodiments, the analyte is a peptide or polypeptide analyte. In some embodiments, the labeling agent comprises an antibody or antigen-binding moiety that binds to the non-nucleic acid analyte. In some embodiments, the labeling agent comprises a reporter oligonucleotide that identifies the analyte. In some embodiments, the labeling agent comprises an optically-detectable label.

In some embodiments, the method comprises analyzing a nucleotide sequence of the analyte, labeling agent, or nucleic acid amplification product at a location in the biological sample or the matrix. In some embodiments, the nucleotide sequence is analyzed by sequential hybridization, sequencing by hybridization, sequencing by ligation, sequencing by synthesis, sequencing by binding, or a combination thereof.

In some aspect, provided herein is a method for processing a biological sample, the method comprising:
a) contacting the biological sample with: (i) a matrix-forming composition comprising a multi-arm monomer or polymer comprising a plurality of arms converging at a central branching point, each arm comprising a functional group R^{A} and the multi-arm monomer or polymer comprises at least one pendant tethering moiety R^{T}; and (ii) a second monomer or polymer comprising at least two functional groups R^{B};
b) forming a matrix embedding the biological sample, wherein the matrix is the product of a polymerization reaction between R^{A} of the multi-arm monomer or polymer and R^{B} of the second monomer or polymer; and
c) attaching the tethering moiety R^{T} to an attachment moiety that is attached to an analyte, labeling agent, or nucleic acid amplification product, or any combination thereof in the biological sample,
thereby attaching the analyte, labeling agent, or nucleic acid amplification product, or any combination thereof to the matrix.

In some embodiments, the biological sample is a fixed and/or permeabilized biological sample. In some embodiments, the biological sample is a tissue sample. In some embodiments, the biological sample is a frozen tissue sample or a fresh tissue sample. In some embodiments, the tissue sample is a tissue slice between about 1 μm and about 50 μm in thickness, optionally wherein the tissue slice is between about 5 μm and about 35 μm in thickness.

In some aspects, provided herein is a hydrogel matrix embedding a biological sample that is the product of a polymerization reaction between:
(i) a multi-arm monomer or polymer comprising a plurality of arms converging at a central branching point, each arm comprising a functional group R^{A}, and
(ii) a second monomer or polymer comprising at least two functional groups R^{B} and a pendant tethering moiety R^{T},
wherein the polymerization reaction is between R^{A} and R^{B}; and
wherein R^{T} is attached to a cognate attachment moiety that is directly or indirectly attached to an analyte, a labeling agent associated with an analyte, or a nucleic acid amplification product, or any combination thereof in the biological sample.

In some embodiments, in the hydrogel matrix embedding a biological sample,
(i) the multi-arm monomer or polymer is a compound according to Formula I:

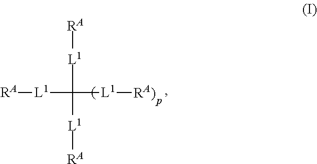

or a salt thereof,
wherein p is 0 or 1;
wherein L¹ is an C₅-C₁₀₀ alkylene optionally interrupted by one or more groups independent selected from the group consisting of —NH—, —O—, —S—, —SO₂—, —N(C₁₋₆ alkyl)-, C₆-C₁₀ aryl, 5- to 6-membered heteroaryl, C₃-C₈ cycloalkyl, and 5- to 6-membered heterocycle, and L¹ is optionally substituted with one or more substituents independently selected from the group consisting of oxo, halo, —OH, —CN, C₁-C₆ alkoxy, C₁-C₆ haloalkoxy, or a side chain of an amino acid;
and
(ii) the second monomer or polymer is a compound according to Formula II:

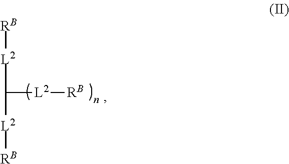

or a salt thereof,
wherein n is 0, 1, or 2;
wherein L² is an C₅-C₁₀₀ alkylene optionally interrupted by one or more groups independent selected from the group consisting of —NH—, —O—, —S—, —SO₂—, —N(C₁₋₆ alkyl)-, a C₆-C₁₀ aryl, 5- to 6-membered heteroaryl, C₃-C₈ cycloalkyl, and 5- to 6-membered heterocycle, and L² is optionally substituted with one or more substituents independently selected from the group consisting of oxo, halo, —OH, —CN, amide, C₁-C₆ alkoxy, C₁-C₆ haloalkoxy, and a side chain of an amino acid;
wherein L² is further attached to one or more tethering moiety R^{T};
wherein R^{A} is capable of reacting with R^{B} to form covalent bond; and R^{T} is substantially unreacted under the condition of R^{A} reacting with R^{B}.

In some embodiments, the second monomer or polymer is of Formula (II-1)

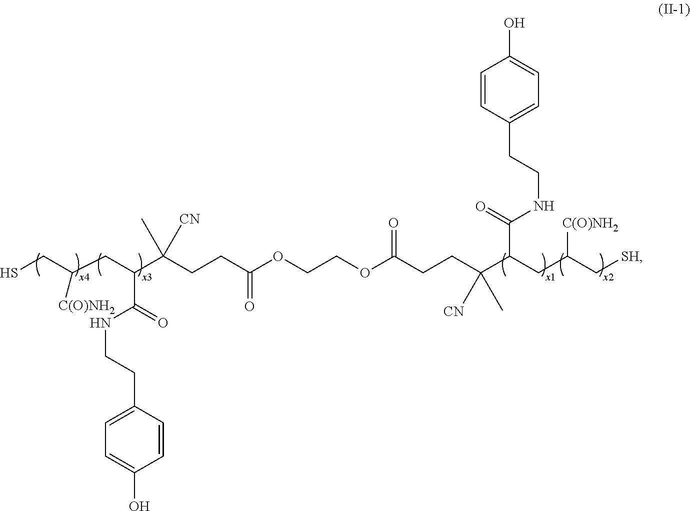

wherein ×1, ×2, ×3, and ×4 are each independently an integer from 1 to 10.

In some embodiments, the second monomer or polymer is of Formula (II-2):

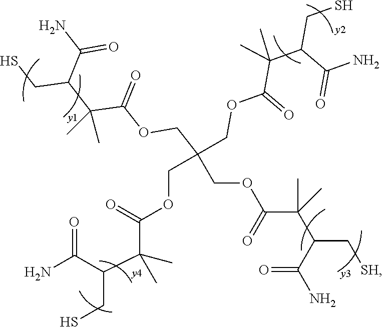

wherein y1, y2, y3, and y4 are each independently an integer from 1 to 10.

In some embodiments, the second monomer or polymer is of Formula (II-3)

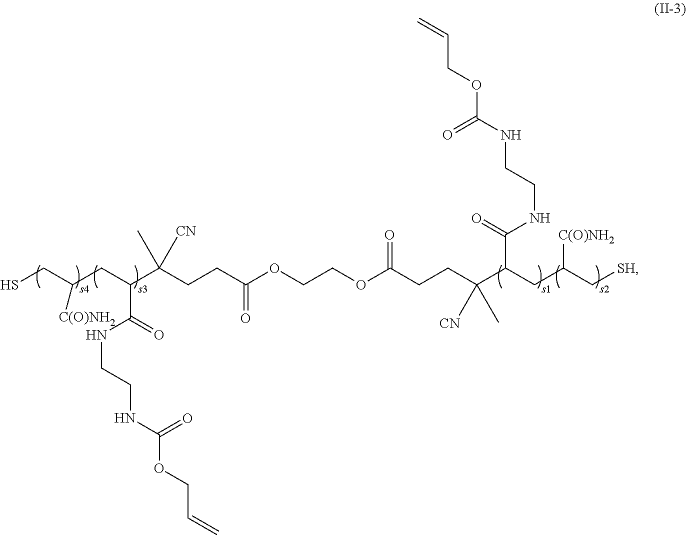

wherein s1, s2, s3, and s4 are each independently an integer from 1 to 10.

In some embodiments, the multi-arm monomer or polymer is of Formula (I-1)

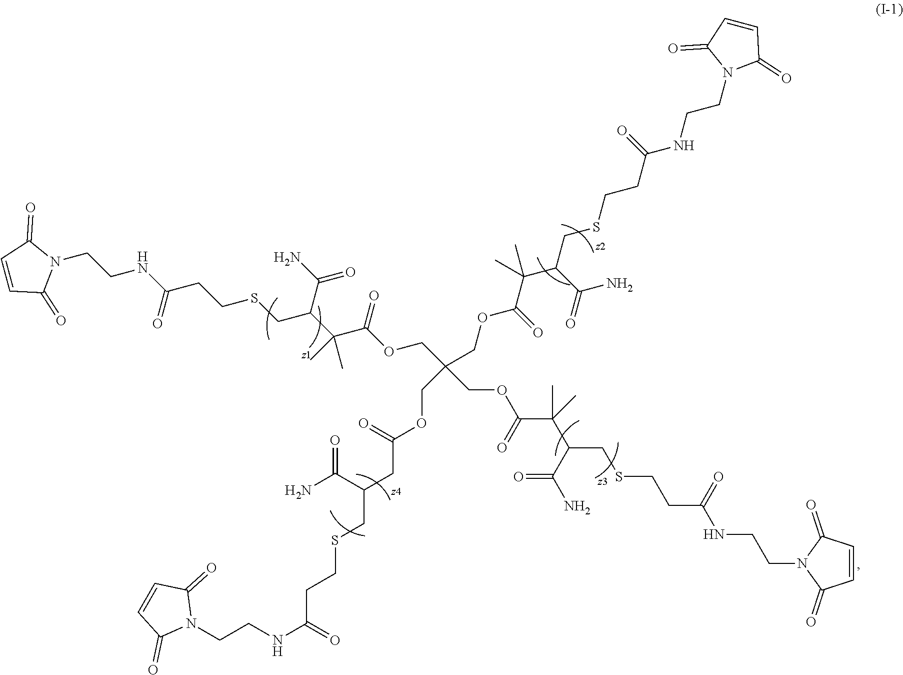

wherein z1, z2, z3, and z4 are each independently an integer from 1 to 10.

In some aspects, provided herein is a polymer matrix subunit according to Formula (II-1)

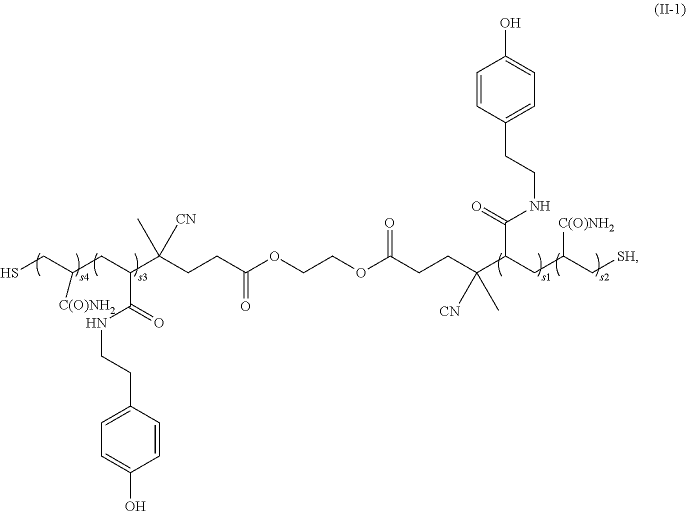

wherein ×1, ×2, ×3, and ×4 are each independently an integer from 1 to 10.

In some embodiments, the second monomer or polymer is of Formula (II-2):

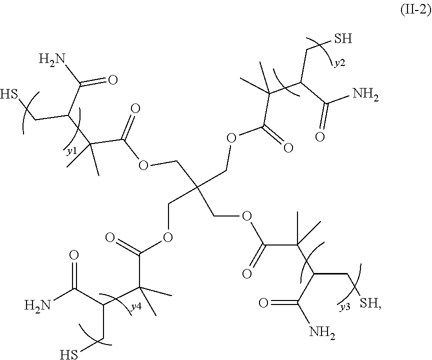

wherein y1, y2, y3, and y4 are each independently an integer from 1 to 10.

In some embodiments, provided herein is a polymer matrix subunit according to Formula (II-3)

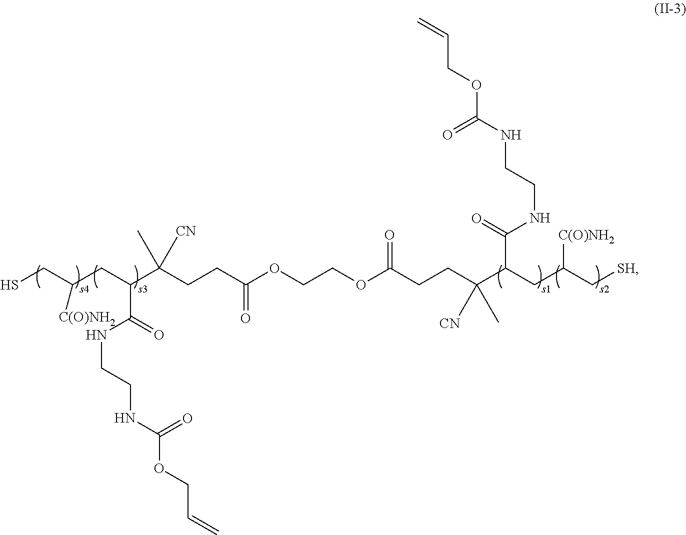

wherein s1, s2, s3, and s4 are each independently an integer from 1 to 10.

In some aspects, provided herein is a polymer matrix subunit according to Formula (I-1)

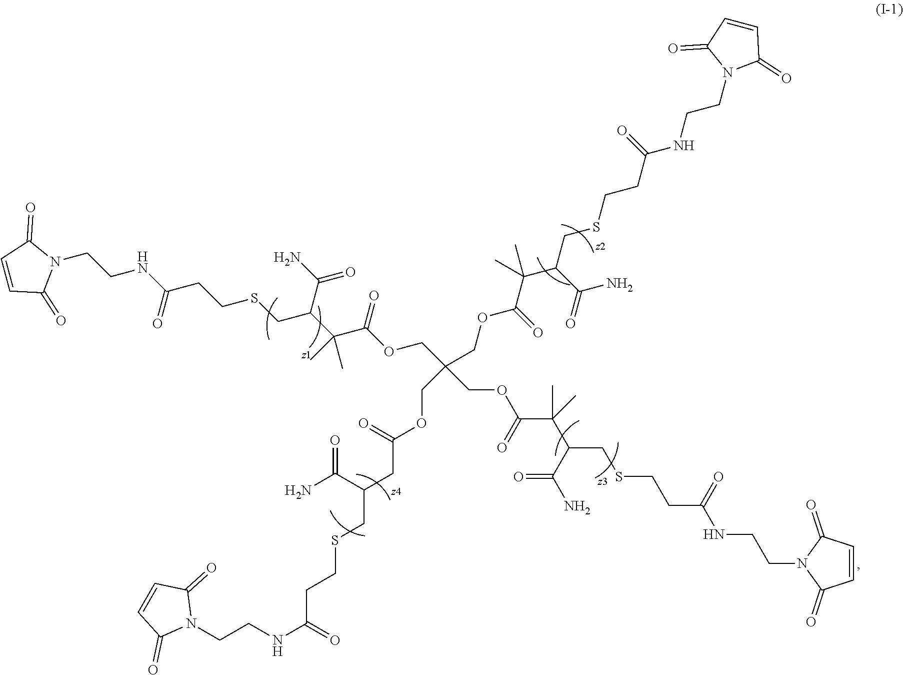

wherein z1, z2, z3, and z4 are each independently an integer from 1 to 10.

In some embodiments, provided herein is a kit for processing a biological, comprising:
(i) a multi-arm monomer or polymer comprising a plurality of arms converging at a central branching point, each arm comprising a functional group R^{A}, and
(ii) a second monomer or polymer comprising at least two functional groups R^{B} and a pendant tethering moiety R^{T},
wherein R^{A} and R^{B} are capable of covalently reacting to crosslink the multi-arm monomer or polymer and the second monomer or polymer without reacting R^{T};
wherein R^{T} is capable of reacting with an attachment moiety using a chemistry orthogonal to the reaction between R^{A} and R^{B}.

### BRIEF DESCRIPTION OF THE DRAWINGS

The drawings illustrate certain features and advantages of this disclosure. These embodiments are not intended to limit the scope of the appended claims in any manner.

FIG. 1 illustrates an example of a method of chemically modifying the analyte, such as an RNA, to enable its tethering on R^{T}.

FIG. 2A, FIG. 2B, and FIG. 2C illustrate examples of a method of forming a matrix embedding the biological sample, wherein the matrix is the product of a polymerization reaction between the R^{A} of the multi-arm monomer or polymer and R^{B} of the second monomer or polymer.

FIGS. 3A-3C illustrate examples of methods of synthesizing second monomers or polymers.

FIG. 4 illustrates an example of a method of synthesizing a multi-arm monomer or polymer.

FIG. 5 illustrates an example of a method of forming a matrix embedding the biological sample, wherein the matrix is the product of a polymerization reaction between the R^{A} of the multi-arm monomer or polymer and R^{B} of the second monomer or polymer.

FIG. 6A and FIG. 6B illustrate other examples of multi-arm monomers or polymers and second monomers or polymers.

FIG. 7A and FIG. 7B illustrate examples of methods of modifying the substrate and further linking the multi-arm monomer or polymer onto the substrate.

FIG. 8 is an example workflow for processing and analyzing a biological sample according to the present disclosure.

FIG. 9 is a schematic illustration of matrix formation using a multi-arm monomer or polymer and second monomer or polymer according to the present disclosure, and attachment of a RCP functionalized with an attachment moiety to a tethering moiety R^{T} of the second monomer or polymer using an orthogonal reaction chemistry.

FIG. 10 is an example workflow of analysis of a biological sample (e.g., a cell or tissue sample) using an opto-fluidic instrument, according to various embodiments.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Provided herein are methods and kits for processing a biological sample. In some embodiments, the biological sample (e.g., tissue sample) comprises highly degraded or fragmented RNA. The methods and kits provided herein can be applied to various applications such as in situ methods. In situ analysis of the identity and spatial localization of RNA requires positional stability of the RNA. However, the preparation of many samples for in situ analysis undergo several harsh processing steps (e.g., formalin-fixed, paraffin-embedded (FFPE) tissues). These steps include baking and deparaffinization, decrosslinking, and permeabilization. In some cases, a vast majority of RNA (e.g., mRNA) can be lost during and after the decrosslinking step.

Hydrogel-based approaches to emerging in situ technologies offer certain advantages including reduced background autofluorescence and improved diffusional parameters. However, most hydrogel-based approaches have notable limitations. For example, many hydrogels suffer from inhomogeneity and have no inherent chemistry to crosslink analytes or products thereof. Some hydrogels made of a random network may have various undesirable structures such as loops, entanglements, dangling chains, and in some cases have non-uniform sizes and concentration throughout the formed matrix. The methods provided herein are intended to overcome many of these shortcomings through the utilization of polymer networks with custom crosslinkers (e.g., networks comprising multi-arm monomers, polymers, or macromonomers), which enable greater network uniformity and the covalent incorporation of analyte via orthogonal chemistries. In some embodiments, enhanced tissue adhesion to the hydrogel and temporally spaced orthogonal chemistries are leveraged to couple the tissue and/or its contents to the hydrogel. The cross-linking multi-arm monomer or polymer and the second monomer or polymer provided herein further enable covalent binding to the analyte or products thereof generated in the sample independent of gelation chemistry.

In some aspects, provided herein are methods for generating a matrix for crosslinking multiple analytes (e.g., different types of analytes) in the same polymer system. Compared to existing hydrogel-based approaches where analytes are linked to end groups, the crosslinking provided herein generates uniform pore size and a resulting uniform network for crosslinking analytes (e.g., or products generated in the matrix). In some embodiments, the crosslinker for capturing analytes or products thereof is different from the crosslinker used for matrix formation (e.g., for forming the hydrogel). In some embodiments, the capturing of analytes or products thereof is performed separately (e.g., in a different step) from matrix formation (e.g., for forming the hydrogel). In some embodiments, the capturing of analytes or products thereof is performed at the same time (e.g., in the same step) as matrix formation (e.g., for forming the hydrogel). In some embodiments, the crosslinking provided comprise compatible chemistries for preparing the biological sample and/or analyzing different analytes.

### II. Methods for Processing a Biological Sample

In one aspect, provided herein is a method for processing a biological sample, the method comprising: a) contacting the biological sample with: (i) a multi-arm monomer or polymer comprising a plurality of arms converging at a central branching point, each arm comprising a functional group R^{A}, and (ii) a second monomer or polymer comprising at least two functional groups R^{B} and a pendant tethering moiety R^{T}; b) forming a matrix embedding the biological sample, wherein the matrix is the product of a polymerization reaction between the R^{A} of the multi-arm monomer or polymer and R^{B} of the second monomer or polymer; and c) attaching R^{T} to an attachment moiety that is directly or indirectly attached to an analyte, a labeling agent associated with an analyte, or nucleic acid amplification product in the biological sample, or any combination thereof, thereby attaching the analyte, labeling agent, or nucleic acid amplification product, or any combination thereof to the matrix.

In some embodiments, “pendant group” may refer to the group that is attached to the molecular moiety that resides between the chain-end functional group (i.e., the crossing linking group) and the central branching point (or nodal point); or if there is no central branching point (e.g., second monomer or polymer), “pendant group” may refer to the group that is attached to the molecular moiety that resides between two functional groups. In some embodiments, the “pendant group” is a side chain of the polymer. For instance, in some embodiments, the multi-arm monomer or polymer is a compound according to Formula I:

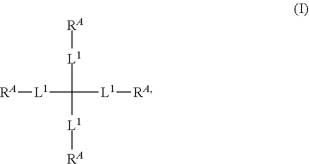

or a salt thereof, wherein the central branching point is the point at which the four -L¹-R^{A} arms converge, and the pendent group may be any substituent or side chain on the molecular moiety that resides between R^{A} and the central branching point. For another instance, in some embodiments, the monomer or polymer is a compound of Formula II:

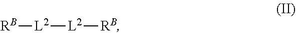

or a salt thereof, wherein there is no central branching point, and the pendant group may be any substituent or side chain on the molecular moiety that resides between the two functional groups R^{B}. In some embodiments, the pendant group on the multi-arm monomer or polymer or on the second monomer or polymer is a pendant tethering moiety R^{T}. In some embodiments, R^{T} is a chemical functional group. In some embodiments, R^{T} does not comprise a peptide.

In some embodiments, the biological sample is contacted with a matrix-forming composition comprising the multi-arm monomer or polymer and the second monomer or polymer. In some embodiments, the biological sample is contacted with the first monomer or polymer and the second monomer or polymer sequentially, in either order. In some embodiments, the forming of the matrix and the attaching of the tethering moiety to the cognate attachment moiety are performed simultaneously. In some embodiments, attaching the tethering moiety to the cognate attachment moiety is performed after the matrix is formed. In some embodiments, the method further comprises clearing the biological sample.

In some embodiments, the method described herein utilizes orthogonal reactions to attach an analyte, labeling agent, or nucleic acid amplification product, or any combination thereof to a matrix. In some embodiments, the cross-linking or polymerization reaction for forming the matrix is orthogonal to the reaction for attaching the analyte, labeling agent, or nucleic acid amplification product, or any combination thereof to a matrix. In some embodiments, when R^{A} reacts with R^{B} to form the matrix, R^{T} is substantially unreacted, such as less than about any of 20%, 10%, 5%, 2%, 1%, or 0.1% of R^{T} is reacted. In some embodiments, when R^{T} reacts with the analyte, labeling agent, or nucleic acid amplification product, or any combination thereof, the matrix formed by the multi-arm monomer or polymer and the second monomer or polymer is substantially unreacted, such as less than about any of 20%, 10%, 5%, 2%, 1%, or 0.1% of the matrix is reacted. In some embodiments, R^{A} and R^{B} can be any pair of complementary groups that are capable of forming a covalent bond, such as a pair of nucleophilic-electrophilic groups or a pair of click functional groups. In some embodiments, R^{T} can be any group that is substantially unreacted when R^{A} reacts with R^{B} to form the matrix. In some embodiments, R^{T} is capable of forming a covalent or non-covalent bond with a group in the analyte, labeling agent, or nucleic acid amplification product, or any combination thereof. In some embodiments, the analyte, labeling agent, or nucleic acid amplification product, or any combination thereof can comprise or be chemically modified to comprise an attachment moiety that is capable of forming a covalent or non-covalent bond with R^{T}. In some embodiments, R^{T} and the attachment moiety in the analyte, labeling agent, or nucleic acid amplification product, or any combination thereof can be selected as a pair, such as a pair of nucleophilic-electrophilic groups, a pair of click functional groups, or a streptavidin/biotin binding pair. In some embodiments, the reaction between the R^{A} and R^{B} and the reaction between the R^{T} and the attachment moiety in the analyte, labeling agent, or nucleic acid amplification product, or any combination thereof are orthogonal. In some embodiments, the reaction between the R^{A} and R^{B} and the reaction between the R^{T} and the attachment moiety associated with the analyte are orthogonal. In some embodiments, R^{A}, R^{B}, R^{T} and the attachment moiety in the analyte, labeling agent, or nucleic acid amplification product, or any combination thereof can be any groups that are configured to have two orthogonal reactions. In some embodiments, this orthogonality can be achieved via different types of reactions, different reaction conditions, different catalysts, or any combination thereof. In some embodiments, the polymerization reaction between R^{A} and R^{B} and the tethering reaction between R^{T} and attachment moiety in the analyte are selected from the group consisting of Horseradish peroxidase (HRP) catalyzed coupling, Michael addition, Diels Alder coupling (e.g., maleimide/furan coupling) ally-thiol coupling, click reaction (e.g., alkyne/azide), acrylamide coupling, Tyrosinase-catalyzed coupling (e.g., coupling of phenol-amine), Transglutaminase-catalyzed coupling (e.g., coupling of thiol-maleimide), Cu-catalyzed click coupling, light-catalyzed coupling (e.g., sulfur/allyl ester coupling and sulfide/norbornene coupling). In some embodiments, the polymerization reaction between R^{A} and R^{B} and the tethering reaction between R^{T} and attachment moiety in the analyte are selected from the group consisting of Horseradish peroxidase (HRP) catalyzed coupling, Michael addition, Diels Alder coupling (e.g., maleimide/furan coupling) ally-thiol coupling, click reaction (e.g., alkyne/azide), acrylamide coupling, Tyrosinase-catalyzed coupling (e.g., coupling of phenol-amine), Transglutaminase-catalyzed coupling (e.g., coupling of thiol-maleimide), Cu-catalyzed click coupling, light-catalyzed coupling (e.g., sulfur/allyl ester coupling and sulfide/norbornene coupling), wherein the polymerization and the tethering reaction are orthogonal.

In some embodiments, the backbone between R^{A} groups in the multi-arm monomer or polymer is any chemical moieties that are chemically compatible with the polymerization and tethering reactions. In some embodiments, the backbone between R^{B} groups in the second monomer or polymer is any chemical moieties that are chemically compatible with the polymerization and tethering reactions.

In some embodiments, the multi-arm monomer or polymer is a 3-arm monomer or polymer. In some embodiments, the multi-arm monomer or polymer is a 4-arm monomer or polymer. In some embodiments, the number of arms in the multi-arm monomer or polymer is optimized in order to change the mesh size. In certain embodiments, tuning the mesh size by changing the number of arms in the multi-arm polymer advantageously allows mesh size adjustment for varying accessibility needs for any given biomolecule of interest (e.g., accessibility of a rolling circle amplification product, RNA, or enzymes or other reagents such as probes that are contacted with the biological sample).

In some embodiments, the multi-arm monomer or polymer is a compound according to Formula I:

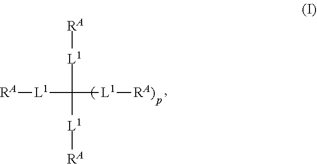

or a salt thereof,
wherein p is 0 or 1;
L¹ is a C₅-C₁₀₀ alkylene optionally interrupted by one or more groups independent selected from the group consisting of —NH—, —O—, —S—, —SO₂—, —N(C₁₋₆ alkyl)-, C₆-C₁₀ aryl, 5- to 6-membered heteroaryl, C₃-C₈ cycloalkyl, and 5- to 6-membered heterocycle, and L¹ is optionally substituted with one or more substituents independently selected from the group consisting of oxo, halo, —OH, —CN, C₁-C₆ alkoxy, C₁-C₆ haloalkoxy, or a side chain of an amino acid.

In some embodiments, L¹ is a C₅-C₅₀ (e.g., C₁₀-C₆₀, C₁₀-C₅₀, C₁₀-C₄₀, C₁₀-C₃₀, or C₁₀-C₂₀) alkylene optionally interrupted by one or more groups independent selected from the group consisting of —NH—, —O—, —S—, —SO₂—, —N(C₁₋₆ alkyl)-, C₆-C₁₀ aryl, 5- to 6-membered heteroaryl, C₃-C₈ cycloalkyl, and 5- to 6-membered heterocycle, and L¹ is optionally substituted with one or more substituents independently selected from the group consisting of oxo, halo, —OH, —CN, C₁-C₆ alkoxy, C₁-C₆ haloalkoxy, or any amino acid side chain. In some embodiments, L¹ comprises or is an unbranched or branched C₅-C₅₀ alkylene, which can be interrupted by 0 to 30 independently selected O, NH, N, S, C₆-C₁₀ aryl, or 5- to 6-membered heteroaryl. In some embodiments, L¹ comprises or is an unbranched and uninterrupted C₁₀-C₅₀ alkylene. In some embodiments, L¹ comprises or is a branched and uninterrupted C₁₀-C₂₀ alkylene. In some embodiments, L¹ comprises or is an unbranched C₁₀-C₂₀ alkylene interrupted by 1 to 8 NH, O, or S. In some embodiments, L¹ comprises or is

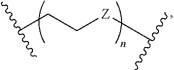

Z is CH₂, O, S; or NH; and n is an integer between 1 and 10. In some embodiments, L¹ is

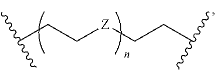

Z is CH₂, O, S; or NH; and n is an integer between 1 and 10 (e.g., any of 1, 2, 3, 4, 5, 6, 7, 8, 9). In some embodiments, L¹ comprises or is an unbranched C₁₀-C₂₀ alkylene interrupted by 1 to 8 oxygen. In some embodiments, L¹ comprises a polyethylene glycol portion or is a polyethylene glycol moiety. In some embodiments, L¹ comprises or is

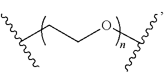

and n is an integer between 1 and 10. In some embodiment, L¹ is

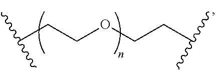

and n is an integer between 1 and 10 (e.g., any of 1, 2, 3, 4, 5, 6, 7, 8, 9). In some embodiments, L¹ comprises an oligoethylene glycol. In some embodiments, L¹ is an oligoethylene glycol moiety. In some embodiments, L¹ comprises or is an unbranched C₅-C₅₀ alkylene interrupted by 0 to 30 sulfurs. In some embodiments, L¹ comprises or is

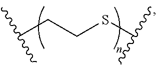

and n is an integer between 1 and 10. In some embodiment, L¹ is

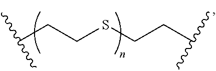

and n is an integer between 1 and 10 (e.g., any of 1, 2, 3, 4, 5, 6, 7, 8, 9). In some embodiments, L¹ comprises or is a branched C₅-C₅₀ alkylene interrupted by 0 to 30 —NH—. In some embodiments, L¹ comprises or is an unbranched C₁-C₁₅₀ alkylene interrupted by 1 to 50 —NH—. In some embodiments, L¹ comprises or is

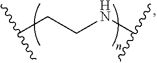

and n is an integer between 1 and 10. In some embodiment, L¹ is

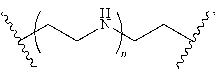

and n is an integer between 1 and 10 (e.g., e.g., any of 1, 2, 3, 4, 5, 6, 7, 8, 9). In some embodiments, L¹ comprises or is a branched C₅-C₅₀ alkylene interrupted by 1 to 30-N—, wherein the —N— is at a branching point. In some embodiments, L¹ comprises or is an unbranched or branched C₅-C₅₀ alkylene interrupted by 0 to 30 independently selected C₆-C₁₂ arylene, for example, any of phenyl or naphthalene. In some embodiments, L¹ comprises or is an unbranched or branched C₁₀-C₅₀ alkylene interrupted by 1 to 30 independently selected 5- to 12-membered heteroarylene, for example, any of pyridine, furan, pyrrole, or thiophene.

In some embodiments, L¹ comprises a PEG unit.

In some embodiments, any of the L¹ can be optionally substituted with one or more substituents independently selected from the group consisting of oxo, halo, —OH, —CN, C₁-C₆ alkoxy, amide, or C₁-C₆ haloalkoxy. In some embodiments, any of the L¹ is optionally substituted with one or more substituents independently selected from the group consisting of oxo, halo, —OH, —CN, C₁-C₆ alkoxy, C₁-C₆ haloalkoxy, or any amino acid side chain.

In some embodiments, L¹ comprises a polyacrylamide unit. In some embodiments, L1 comprises

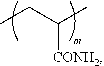

wherein m is any of 1, 2, 3, 4, 5, 6, 7, 8, 9, or 10.

In some embodiments, L¹ comprises a peptide. In some embodiments, the peptide comprises any of 2, 3, 4, 5, 6, 7, 8, 9, or 10 amino acids.

In some embodiments, the second monomer or polymer is a monomer or polymer with two functional groups and without a central branching point. In some embodiments, the second monomer or polymer is a linear homotelechelic monomer or polymer. In some embodiments, “telechelic” may refer to functional chain-ends, such as the R^{B} in the second monomer or polymer, and in some embodiments, the plurality of functional chain-ends, such as the R^{B} in the second monomer or polymer, are the same functional group. In some embodiments, the second monomer or polymer is a linear homotelechelic monomer without repeating units. In some embodiments, the second monomer or polymer is a linear homotelechelic polymer comprising a repeating unit.

In some embodiments, the second monomer or polymer comprises a plurality of arms converging at a central branching point, each arm comprising a R^{B}. In some embodiments, the second monomer or polymer has three arms converging at a central branching point. In some embodiments, the second monomer or polymer has four arms converging at a central branching point.

In some embodiments, the second monomer or polymer is a compound according to Formula II:

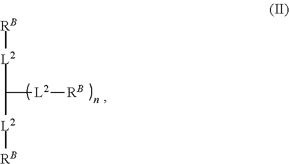

or a salt thereof,
wherein n is 0, 1, or 2;
wherein L² is a C₅-C₁₀₀ alkylene optionally interrupted by one or more groups independent selected from the group consisting of —NH—, —O—, —S—, —SO₂—, —N(C₁₋₆ alkyl)-, a C₆-C₁₀ aryl, 5- to 6-membered heteroaryl, C₃-C₈ cycloalkyl, and 5- to 6-membered heterocycle, and L² is optionally substituted with one or more substituents independently selected from the group consisting of oxo, halo, —OH, —CN, amide, C₁-C₆ alkoxy, C₁-C₆ haloalkoxy, and a side chain of an amino acid;
wherein L² is further attached to one or more tethering moiety R^{T};
wherein R^{A} is capable of reacting with R^{B} to form the matrix of b); and
wherein R^{T} is substantially unreacted when R^{A} reacts with R^{B} to form the matrix in b).

In some embodiments, L² does not comprise a peptide backbone. In some embodiments, L² is a protease-insensitive linker. In some embodiments, L² is a C₅-C₁₀₀ (e.g., C₁₀-C₆₀, C₁₀-C₅₀, C₁₀-C₄₀, C₁₀-C₃₀, or C₁₀-C₂₀) alkylene optionally interrupted by one or more groups independent selected from the group consisting of —NH—, —O—, —S—, —SO₂—, —N(C₁₋₆ alkyl)-, C₆-C₁₀ aryl, 5- to 6-membered heteroaryl, C₃-C₈ cycloalkyl, and 5- to 6-membered heterocycle, and L² is optionally substituted with one or more substituents independently selected from the group consisting of oxo, halo, —OH, —CN, C₁-C₆ alkoxy, C₁-C₆ haloalkoxy, or any amino acid side chain. In some embodiments, L² comprises or is an unbranched or branched C₅-C₅₀ alkylene, which can be interrupted by 0 to 30 independently selected O, NH, N, S, C₆-C₁₀ aryl, or 5- to 6-membered heteroaryl. In some embodiments, L² comprises or is an unbranched and uninterrupted C₅-C₅₀ alkylene. In some embodiments, L² comprises or is a branched and uninterrupted C₅-C₂₀ alkylene. In some embodiments, L² comprises or is an unbranched C₁₀-C₂₀ alkylene interrupted by 0 to 8 NH, O, or S. In some embodiments, L² comprises or is

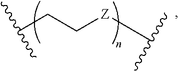

Z is CH₂, O, S; or NH; and n is an integer between 1 and 10. In some embodiments, L² is

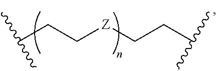

Z is CH₂, O, S; or NH; and n is an integer between 1 and 10 (e.g., any of 1, 2, 3, 4, 5, 6, 7, 8, 9). In some embodiments, L² comprises or is an unbranched C₁₀-C₂₀ alkylene interrupted by 1 to 8 oxygen. In some embodiments, L² comprises a polyethylene glycol portion or is a polyethylene glycol moiety. In some embodiments, L² comprises or is

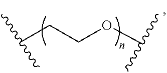

and n is an integer between 1 and 10. In some embodiment, L² is

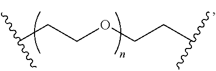

and n is an integer between 1 and 10 (e.g., any of 1, 2, 3, 4, 5, 6, 7, 8, 9). In some embodiments, L² comprises an oligoethylene glycol. In some embodiments, L² is an oligoethylene glycol moiety. In some embodiments, L² comprises or is an unbranched C₅-C₅₀ alkylene interrupted by 1 to 30 sulfurs. In some embodiments, L² comprises or is

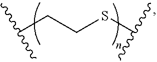

and n is an integer between 1 and 10. In some embodiment, L² is

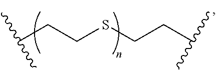

and n is an integer between 1 and 10 (e.g., any of 1, 2, 3, 4, 5, 6, 7, 8, 9). In some embodiments, L² comprises or is a branched C₅-C₅₀ alkylene interrupted by 1 to 30 —NH—. In some embodiments, L² comprises or is an unbranched C₅-C₅₀ alkylene interrupted by 1 to 50 —NH—. In some embodiments, L² comprises or is

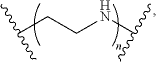

and n is an integer between 1 and 10. In some embodiment, L² is

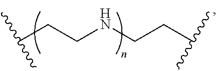

and n is an integer between 1 and 10 (e.g., e.g., any of 1, 2, 3, 4, 5, 6, 7, 8, 9). In some embodiments, L² comprises or is a branched C₅-C₅₀ alkylene interrupted by 0 to 30 —N—, wherein the —N— is at a branching point. In some embodiments, L² comprises or is an unbranched or branched C₅-C₅₀ alkylene interrupted by 0 to 30 independently selected C₆-C₁₂ arylene, for example, any of phenyl or naphthalene. In some embodiments, L² comprises or is an unbranched or branched C₁₀-C₅₀ alkylene interrupted by 0 to 30 independently selected 5- to 12-membered heteroarylene, for example, any of pyridine, furan, pyrrole, or thiophene.

In some embodiments, the second monomer or polymer comprises a poly(ethylene glycol). In some embodiments, L² comprises a PEG unit. In some embodiments L² comprises about 2 to about 20 (e.g., about any of 2 to 18, 2 to 15, 2 to 12, 2 to 10, 4 to 10, 5 to 10, 5, 6, 7, 8, 9) ethylene glycol units.

In some embodiments, any of the L² is optionally substituted with one or more substituents independently selected from the group consisting of oxo, halo, —OH, —CN, amide, C₁-C₆ alkoxy, C₁-C₆ haloalkoxy, and a side chain of an amino acid. In some embodiments, L² is optionally substituted with one or more amide group. In some embodiments, the second monomer or polymer comprises a polyacrylamide. In some embodiments, L² comprises a polyacrylamide unit. In some embodiments, L² comprises

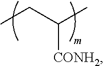

wherein m is any of 1, 2, 3, 4, 5, 6, 7, 8, 9, or 10.

In some embodiments, the second monomer or polymer comprises a peptide. In some embodiments, L² comprises a peptide. In some embodiments, the peptide is between about 5 and about 20 amino acid residues in length. In some embodiments, the peptide comprises any of 2, 3, 4, 5, 6, 7, 8, 9, or 10 amino acids. In some embodiments, both the N-terminal and C-terminal amino acid residues of the peptide comprise the R^{B}. In some embodiments, the peptide comprises cysteine or lysine. In some embodiments, the second monomer or polymer is a peptide comprising a pendant alkyne (R^{T}) and terminal cysteine residues (e.g., for attachment of the cysteine residues to a maleimide R^{A} moiety in a thiol-maleimide reaction). In some embodiments, the second monomer or polymer is a peptide comprising a pendant alkene (R^{T}) and terminal lysine-alkyne residues (e.g., for attachment of the lysine-alkyne to an azide R^{A} moiety, e.g., in an alkyne-azide cycloaddition (CuAAC) reaction). In some embodiments, the second monomer or polymer is a peptide comprising a pendant phenol moiety (R^{T}) and terminal cysteine residues (e.g., for attachment of the cysteine residues to a maleimide R^{A} moiety in a thiol-maleimide reaction). In some embodiments, the second monomer or polymer is a peptide comprising a pendant amide moiety (R^{T}) and terminal cysteine residues (e.g., for attachment of the cysteine residues to a maleimide R^{A} moiety in a thiol-maleimide reaction).

In some embodiments, L² is further attached to one or more tethering moiety R^{T}. In some embodiments, R^{T} is pendant on L². In some embodiments, the pendant tethering moiety R^{T} is attached to an amino acid residue between two R^{B}. In some embodiments, the second monomer or polymer comprises at least two pendant tethering moieties R^{T}, each of which is independently attached to an independently selected amino acid residue between two R^{B}.

In some embodiments, R^{A} is a nucleophilic group and R^{B} is an electrophilic group; or R^{A} is an electrophilic group and R^{B} is a nucleophilic group; or R^{A} and R^{B} are a click chemistry pair.

In some embodiments, one of R^{A} and R^{B} comprises or is a nucleophilic group and the other one of R^{A} and R^{B} comprises or is an electrophilic group. In some embodiments, the nucleophilic group is an amine moiety, an amide moiety, an alcohol moiety, a thiol moiety, a cyano moiety, an ylide moiety, a hydrazide, a hydroxylamine, a hydrazine, a thiosemicarbazone, a hydrazine carboxylate, or an arylhydrazide, or any combination thereof. In some embodiments, the nucleophilic group is an amine moiety, an amide moiety, an alcohol moiety, a thiol moiety, a cyano moiety, or an ylide moiety. In some embodiments, the nucleophilic group is a thiol moiety (e.g., —SH). In some embodiments, the electrophilic group is a carbonyl, an amide, an acrylamide, a maleimide, haloacetamide, or NHS ester.

In some embodiments, R^{A} comprises or is a click functional group. In some embodiments, R^{B} comprises or is a click functional group. Suitable click functional groups may include functional groups compatible with a nucleophilic addition reaction, a cyclopropane-tetrazine reaction, a strain-promoted azide-alkyne cycloaddition (SPAAC) reaction, an alkyne hydrothiolation reaction, an alkene hydrothiolation reaction, a strain-promoted alkyne-nitrone cycloaddition (SPANC) reaction, an inverse electron-demand Diels-Alder (IED-DA) reaction, a cyanobenzothiazole condensation reaction, an aldehyde/ketone condensation reaction, and Cu(I)-catalyzed azide-alkyne cycloaddition (CuAAC) reaction. In some embodiments, the R^{A} and R^{B} comprise or are any functional group involved in click reactions. In some embodiments, such click reactions may involve (i) azido and cyclooctynyl; (ii) azido and alkynyl; (iii) tetrazine and dienophile; (iv) thiol and alkynyl; (v) cyano and amino thiol; (vi) nitrone and cyclooctynyl; or (vii) cyclooctynyl and nitrone. It should be recognized that in instances in which R^{A} comprises or is a click functional group, R^{B} comprises the complementary click functional group to that of the attachment moiety. For example, in some embodiments, R^{A} comprises or is an azide moiety and R^{B} comprises a complementary alkyne moiety, or vice versa.

In some embodiments, R^{T} comprises or is any of the nucleophilic groups provided herein, provided that R^{T} is substantially unreacted when R^{A} reacts with R^{B} to form the matrix in step b). In some embodiments, R^{T} comprises or is any of the electrophilic groups provided herein, provided that R^{T} is substantially unreacted when R^{A} reacts with R^{B} to form the matrix in step b). In some embodiments, R^{T} comprises or is any of the click functional groups provided herein, provided that R^{T} is substantially unreacted when R^{A} reacts with R^{B} to form the matrix in step b). In some embodiments, R^{T} comprises or is an alkenyl, alkynyl, allyl or vinyl moiety, ally ester moiety, an acrylamide moiety, an amide moiety, an alcohol moiety, a polyol moiety, a furan moiety, a maleimide moiety, a norbornene moiety, a thiol moiety, a sulfide moiety, a phenol moiety, a urethane moiety, a cyano moiety, an amino moiety, an isocyanate moiety, an isothiocyanate moiety, an ether moiety, a dextran moiety, or an alginate moiety. In some embodiments, R^{T} comprises or is thiol, phenol, amino, hydrazide, hydroxylamine, hydrazine, thiosemicarbazone, hydrazine carboxylate, or arylhydrazide. In some embodiments, R^{T} comprises or is a phenol moiety. In some embodiments, R^{T} comprises or is an acrylamide moiety. In some embodiments, R^{T} comprises or is an amide moiety. In some embodiments, R^{T} comprises or is an alkyne or a alkene moiety. In some embodiments, R^{T} comprises or is an azide moiety. In some embodiments, R^{T} comprises or is an allyl moiety. In some embodiments, R^{T} comprises or is a carbamate moiety. In some embodiments, R^{T} comprises or is

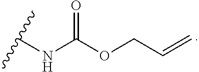 [SMILES: *NC(=O)OCC=C]

In some embodiments, R^{T} comprises a phenol group and the attachment moiety comprises an amine group. In some embodiments, R^{T} comprises an amine group and the attachment moiety comprises a phenol group. In certain embodiments, attaching R^{T} to the attachment moiety comprises performing a tyrosinase reaction. In some embodiments, the attachment moiety is in a primer that is contacted with the biological sample (e.g., a primer that hybridizes to circular or circularized probe in the biological sample to perform rolling circle amplification. In some embodiments, the attachment moiety is incorporated into an amplification or extension product in the biological sample. In certain embodiments, the biological sample is contacted with one or more modified nucleotides functionalized with the attachment moiety, and the one or more modified nucleotides comprising the attachment moiety is/are incorporated into an amplification or extension product in the biological sample. In some cases, the amplification product is attached to the matrix by attaching the attachment moiety to R^{T}.

In some embodiments, (i) R^{T} comprises an alkene group and the attachment moiety comprises a thiol group, or (ii) R^{T} comprises a thiol group and the attachment moiety comprises an alkene group, optionally wherein attaching R^{T} to the attachment moiety comprises performing a thiol-ene addition in the presence of ultraviolet irradiation.

In some embodiments, (i) R^{T} comprises an amide group and the attachment moiety comprises an amine group, or (ii) R^{T} comprises an amine group and the attachment moiety comprises an amide group, optionally wherein attaching R^{T} to the attachment moiety comprises performing amide-amine coupling with transglutaminase.

In some embodiments, (i) R^{T} comprises an azide group and the attachment moiety comprises an alkyne group, or (ii) R^{T} comprises an alkyne group and the attachment moiety comprises an azide group, optionally wherein attaching R^{T} to the attachment moiety comprises performing a copper-ion-catalyzed azide-alkyne cycloaddition reaction (CuAAC reaction).

In some embodiments, R^{T} is directly attached to L² via a covalent bond. In some embodiments, R^{T} is attached to L² via a linker L³. In some embodiments, L³ comprises any of the linking moieties provided herein, such as L¹ and L². In some embodiments, L³ is a C₁-C₁₀ (e.g., C₁-C₈, C₁-C₆, or C₁-C₄)alkylene optionally interrupted by one or more groups independent selected from the group consisting of —NH—, —O—, —S—, —SO₂—, —N(C₁₋₆ alkyl)-, C₆-C₁₀ aryl, 5- to 6-membered heteroaryl, C₃-C₈ cycloalkyl, and 5- to 6-membered heterocycle, and L³ is optionally substituted with one or more substituents independently selected from the group consisting of oxo, halo, —OH, —CN, C₁-C₆ alkoxy, C₁-C₆ haloalkoxy, or any amino acid side chain. In some embodiments, L³ comprises an amide group. In some embodiments, L³ is

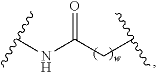

wherein w is 0, 1, 2, 3, 4, or 5, one wavy line denotes the attachment point to L², and the other wavy line denotes the attachment point to R^{T}.

In some embodiments, R^{A} is a thiol group and R^{B} is a maleimide group, or wherein R^{A} is a maleimide group and R^{B} is a thiol group. In some embodiments, the polymerization reaction is a thiol-maleimide Michael coupling reaction.

In some embodiments of Formula (II), n is 0, and R^{B} comprises-SH. In some embodiments, R^{T} comprises phenol, amide, or a combination thereof. In some embodiments, the second monomer or polymer is of Formula (II-1)

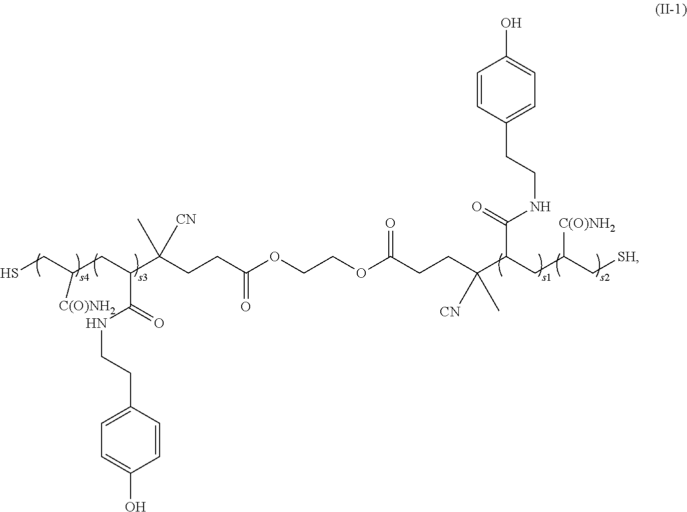

wherein ×1, ×2, ×3, and ×4 are each independently an integer from 1 to 10.

In some embodiments, n is 2 and R^{B} comprises-SH. In some embodiments, the second monomer or polymer is of Formula (II-2):

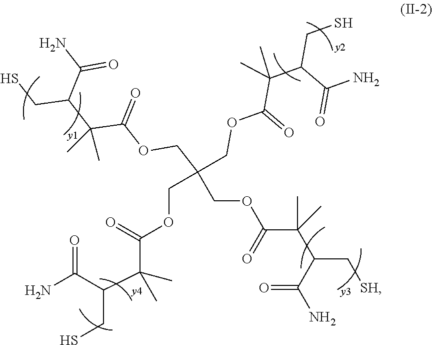

wherein y1, y2, y3, and y4 are each independently an integer from 1 to 10.

In some embodiments, the compound of formula II (e.g., a compound of formula II-2) can be prepared using a reversible-addition-fragmentation chain transfer (RAFT) polymerization reaction. For instance, RAFT polymerization can be carried out by reacting a monomer with a chain transfer agent (CTA) in the presence of a radical initiator. In particular embodiments, the CTA is a 4-armed functionalized CTA (4f-CTA), wherein each arm comprises a thiocarbonylthio moiety. In some embodiments, the 4f-CTA includes one or more dithioester moieties, dithiocarbamate moieties, trithiocarbonate moieties, or xanthates. In some embodiments, the monomer reacting with the 4f-CTA is an acrylamide, styrene, acrylate, methacrylate, metacrylamide, vinyl ester or vinyl amide.

In some embodiments, the radical initiator is an azo polymerization initiator. In some embodiments, the radical initiator is azobisisobutyronitrile (AIBN) or 4,4′-azobis(4-cyanovaleric acid) (ACVA), In some embodiments, the radical initiator has a structure depicted below:

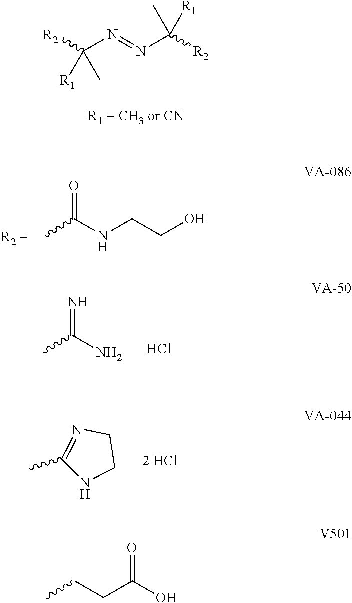

In some embodiments, the radical initiator is a photocatalyst (see, for example, Lee et al., Chem. Soc. Rev. 52:3035, 2023, the entire contents of each of which are incorporated herein by reference.) In some embodiments, the photocatalyst is Ir(ppy)₃. In some embodiments, the photocatalyst is Ru(bpy)₃Cl₂. In some embodiments, the photocatalyst is ZnTPP. In some embodiments the photocatalyst is Eosin Y.

Particular embodiments of 4-armed CTAs are provided below:

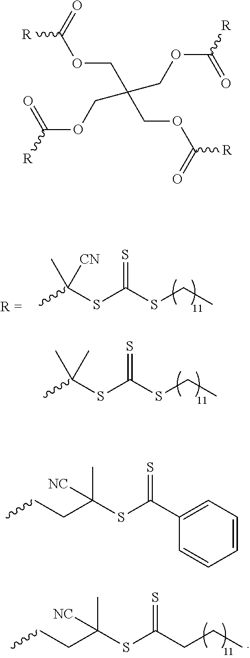

To generate the compound of formula II (e.g., a compound of II-2), a monomer (e.g., acrylamide monomer) can be reacted with a 4f-CTA in the presence of an initiator in DMSO at an elevated temperature for a sufficient time (e.g., at least about 6 hours to about 24 hours) to generate the RAFT polymerization product. In some instances, the reaction with the 4f-CTA is performed for at least 6 hours, at least 8 hours, at least 10 hours, at least 12 hours, at least 14 hours, at least 16 hours, at least 18 hours, or at least 20 hours. In some instances, the reaction with the 4f-CTA is performed for about 5 hours to about 24 hours, about 6 hours to about 24 hours, about 12 hours to about 24 hours, about 16 hours to about 24 hours, or about 20 hours to about 24 hours. In some embodiments, the compound of formula II (e.g., a compound of II-2), a monomer (e.g., acrylamide monomer) is reacted with a 4f-CTA under inert conditions (e.g., in the presence of argon or nitrogen).

A particular example is shown in the schematic below, where an acrylamide monomer is reacted with a 4f-CTA to afford the RAFT polymerization product, which is reacted with tributylphosphine (PBu₃) and a primary amine to afford a compound of formula II. In some embodiments the primary amine is an alkyl amine. In some such embodiments, the alkyl amine is butylamine or hexylamine.

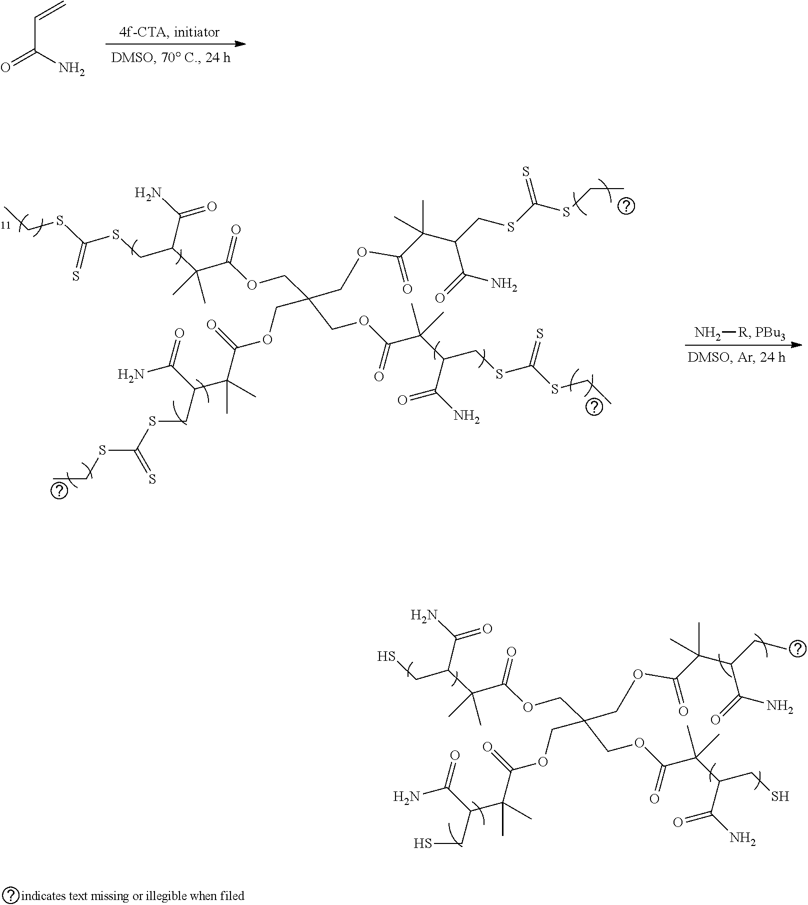

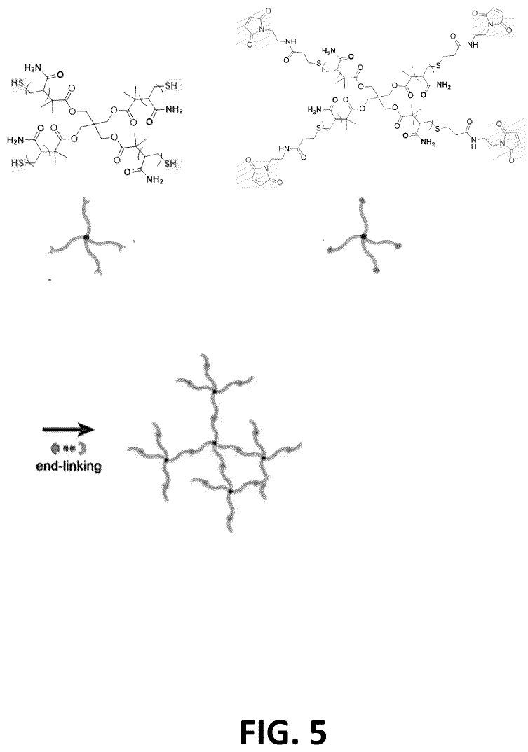

In some embodiments, the multi-arm monomer or polymer is according to Formula (I-1), and the second monomer or polymer is according to Formula (II-2). In some embodiments, the multi-arm monomer or polymer is according to Formula (I-1) wherein z1, z2, z3, and z4 are each independently an integer from 1 to 10, and the second monomer or polymer is according to Formula (II-2) wherein y1, y2, y3, and y4 are each independently an integer from 1 to 10. In some embodiments, the multi-arm monomer or polymer according to Formula (I-1) and the second monomer or polymer according to Formula (II-2) are polymerized together using thiol-maleimide gelation chemistry. In some aspects, the use of two different multi-arm polymers (e.g., 4-arm polymers) provides a high degree of uniformity in the resulting matrix. In some aspects, both multi-arm monomers or polymers comprise pendant tethering moieties. In some embodiments, each arm of each multi-arm monomer or polymer comprises a pendant tethering moiety. In some embodiments, the pendant tethering moiety is an amide moiety (e.g., as in Formula (I-1) and Formula (II-2). FIG. 5 illustrates an example of a method of matrix formation using a multi-arm monomer or polymer according to Formula (I-1) and a second monomer or polymer is according to Formula (II-2).

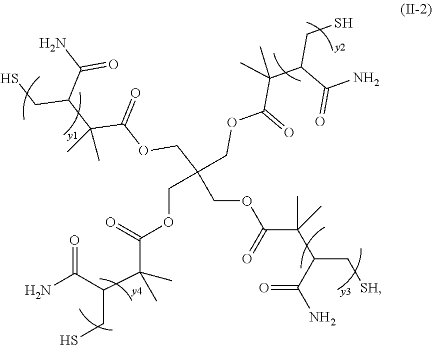

wherein y1, y2, y3, and y4 are each independently an integer from 1 to 10.

In some embodiments, R^{A} comprises a maleimide group. In some embodiments, the multi-arm monomer or polymer is of Formula (I-1)

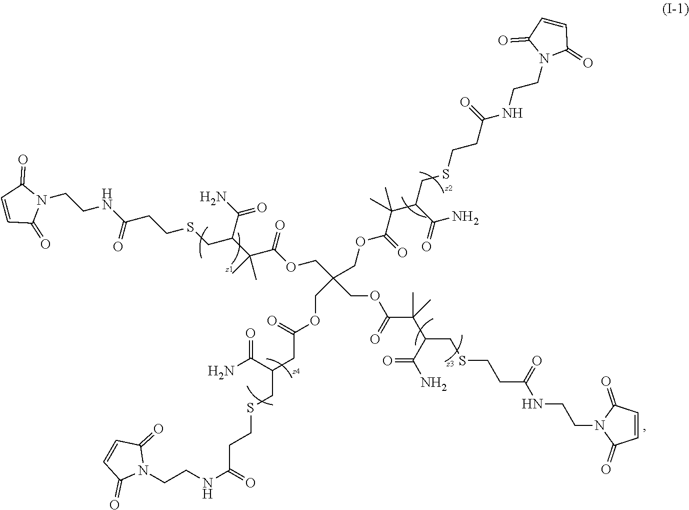

wherein z1, z2, z3, and z4 are each independently an integer from 1 to 10.

In some embodiments, the second monomer or polymer is of Formula (II-3)

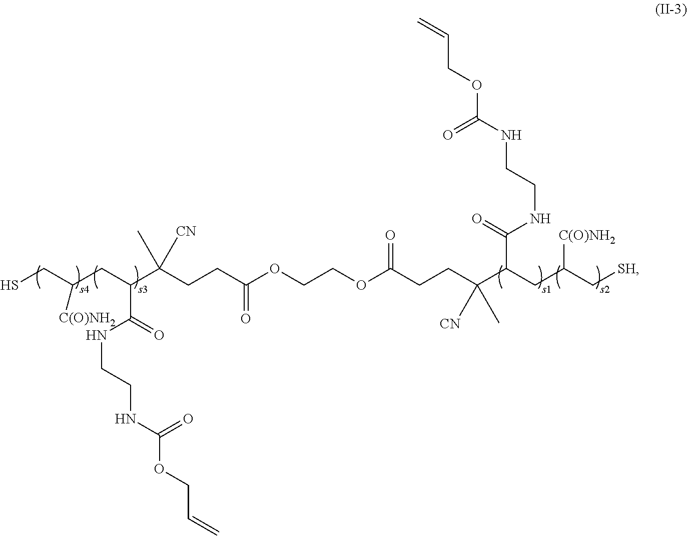

wherein s1, s2, s3, and s4 are each independently an integer from 1 to 10.

In some embodiments, the second monomer or polymer is of Formula (II-4)

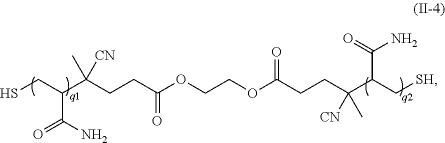

wherein q1 and q2 are each independently an integer from 1 to 10.

In some embodiments, R^{A} comprises a maleimide group. In some embodiments, the multi-arm monomer or polymer is of Formula (I-1)

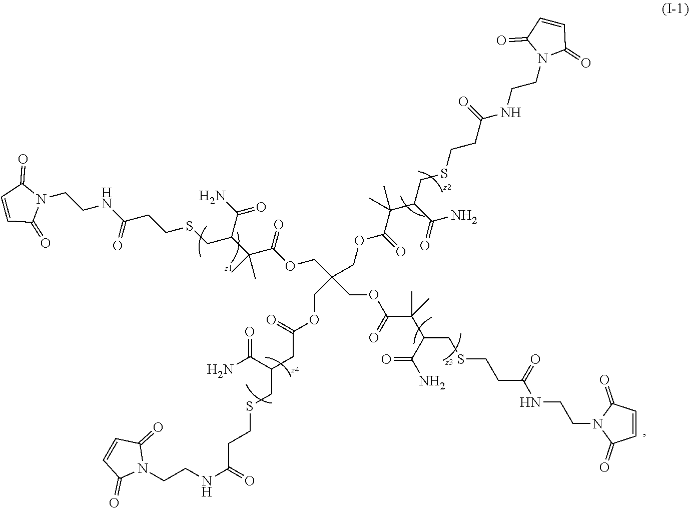

wherein z1, z2, z3, and z4 are each independently an integer from 1 to 10.

In some embodiments, the multi-arm monomer or polymer is of Formula (I-2)

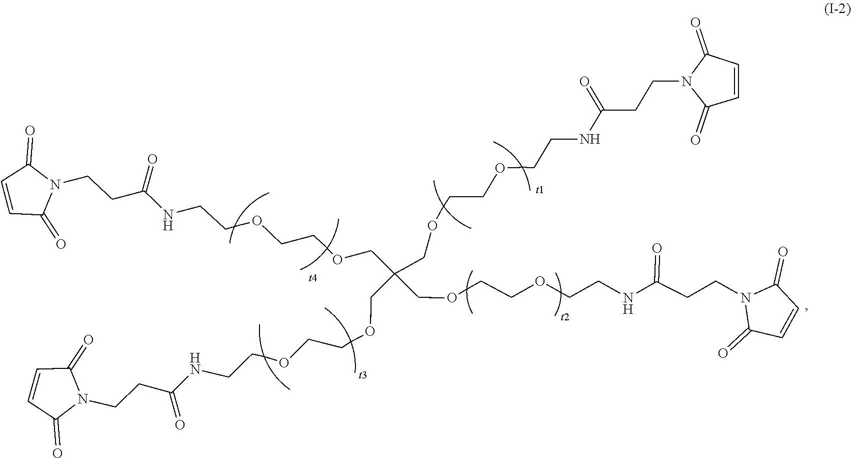

wherein t1, t2, t3, and t4 are each independently an integer from 1 to 10.

In some embodiments, R^{A} is an azide group and R^{B} is an alkyne group, or wherein R^{A} is an alkyne group and R^{B} is an azide group. In some embodiments, the polymerization reaction is a copper-ion-catalyzed azide-alkyne cycloaddition reaction (CuAAC reaction). In some embodiments, the biological sample is on an alkyne-modified substrate.

In some embodiments, R^{A} is a N-hydroxysuccinimide ester (NHS) group and R^{B} is a primary amine group, or wherein R^{A} is a primary amine group and R^{B} is a NHS group. In some embodiments, the polymerization reaction is performed at a pH between about 7 and about 9.

In some embodiments, the second monomer or polymer is of Formula (II-5)

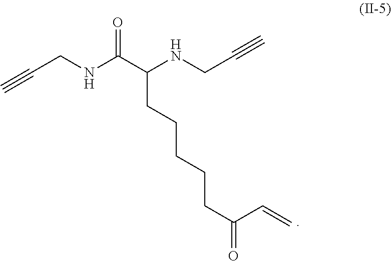 [SMILES: C#CCNC(=O)C(CCCCCC(=O)C=C)NCC#C]

In some embodiments, the second monomer or polymer is of Formula (II-5)

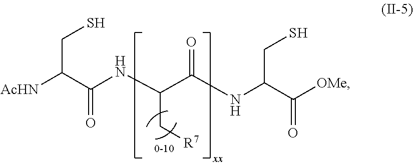

wherein each R^{T} is independently selected from the group consisting of H,

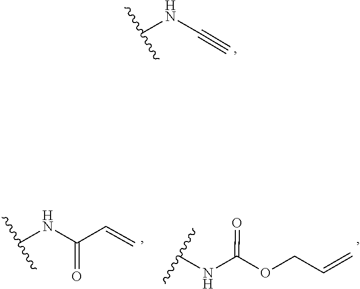

phenol, and —C(O)NH₂, and xx is an integer from 1 to 20, such as an integer from 1 to 10, from 10 to 20, or from 5 to 15. In some embodiments, at least one R^{T} is

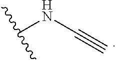 [SMILES: *NC#C]

In some embodiments, at least one R^{T} is

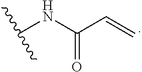 [SMILES: *NC(=O)C=C]

In some embodiments, at least one R^{T} is

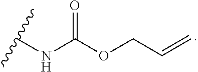 [SMILES: *NC(=O)OCC=C]

In some embodiments, R^{A} comprises an azide group. In some embodiments, the multi-arm monomer or polymer is of Formula (I-3)

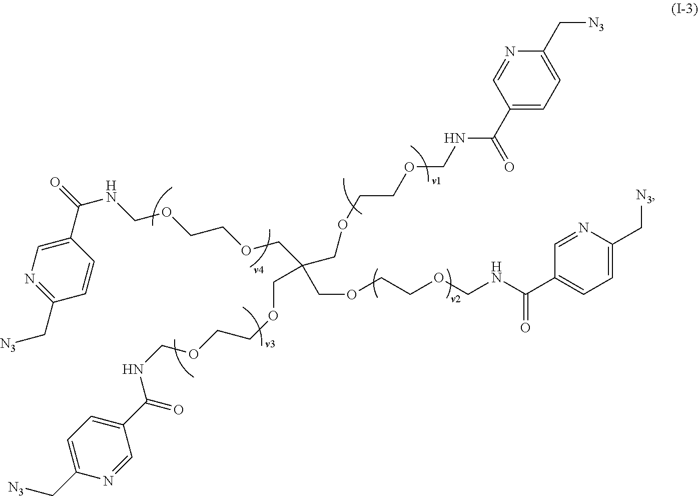

wherein v1, v2, v3, and v4 are each independently an integer from 1 to 10.

In some embodiments, one of the multi-arm monomer or polymer and the second monomer or polymer is of Formula (I-6) and the other one of the multi-arm monomer or polymer and the second monomer or polymer is of Formula (I-7):

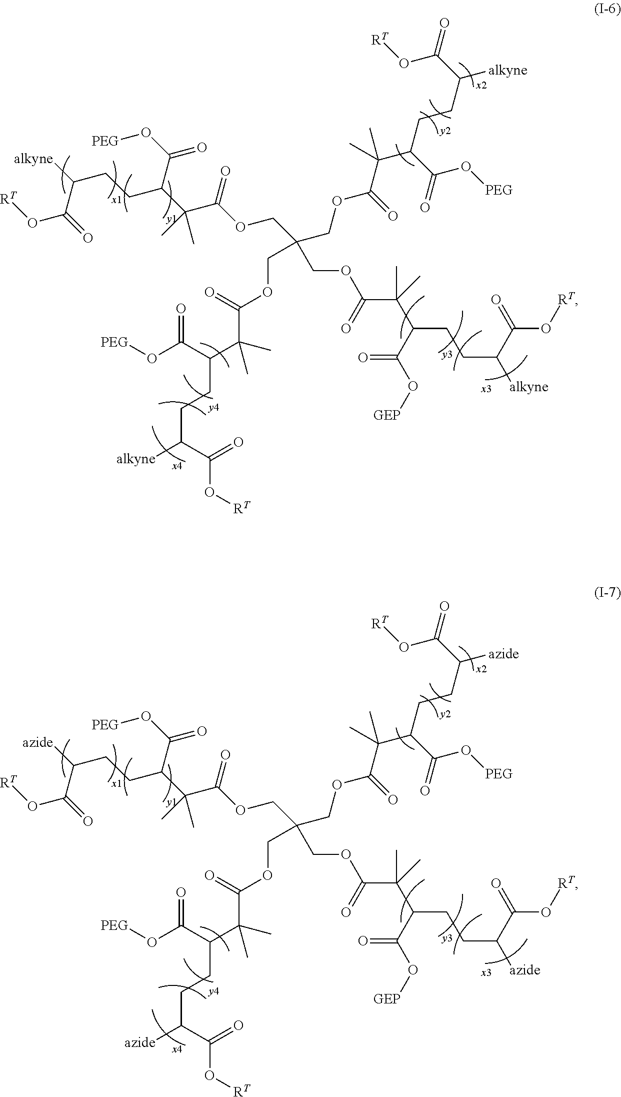

wherein ×1, ×2, ×3, ×4, y1, y2, y3, and y4 are each independently an integer from 1 to 10.

In some embodiments, the multi-arm monomer or polymer is of Formula (I-6) and the second monomer or polymer is of Formula (I-7). In some embodiments, the second monomer or polymer is of Formula (I-6) and the multi-arm monomer or polymer is of Formula (I-7).

In some embodiments, the multi-arm monomer or polymer (e.g., Formula (I-3) comprises at least one pendant tethering moiety R^{T} for attaching to an analyte, labeling agent, or nucleic acid amplification product. In some embodiments, the second monomer or polymer is a second multi-arm monomer or polymer. In some embodiments, the multi-arm monomer or polymer comprises 3, 4, or more arms, and each arm comprises at least one pendant tethering moiety R^{T}. In some embodiments, the second multi-arm monomer or polymer comprises 3, 4, or more arms, and each arm comprises at least one pendant tethering moiety R^{T}.

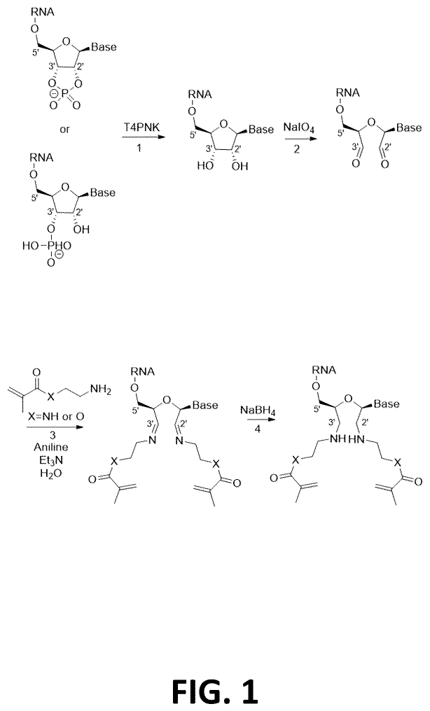

In some embodiments, the method described herein comprises the components or processes illustrated in FIG. 1 to FIG. 6.

FIG. 1 illustrates an example of a method of chemically modifying the analyte or a product thereof, (e.g., RNA) with an attachment moiety for tethering to R^{T}. In some embodiments, the

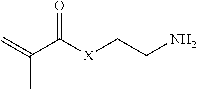

is replaced by any

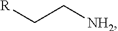

wherein the R is an attachment moiety selected based on the selection of an orthogonal gelation chemistry. In some embodiments, candidate examples of the attachment moiety (R) orthogonal gelation chemistries include, but are not limited to the pairs in the following table:

| R | Orthogonal gelation chemistry |
|---|---|
| acrylamide | radical |
| phenol | phenol (HRP) |
| azide/alkyne | alkyne/azide (Click) |
| norbornene/sulfide | sulfide/norbornene (UV) |
| Furan/maleimide | maleimide/furan (Diels Alder) |
| Allyl ester/sulfur | sulfur/allyl ester (UV) |

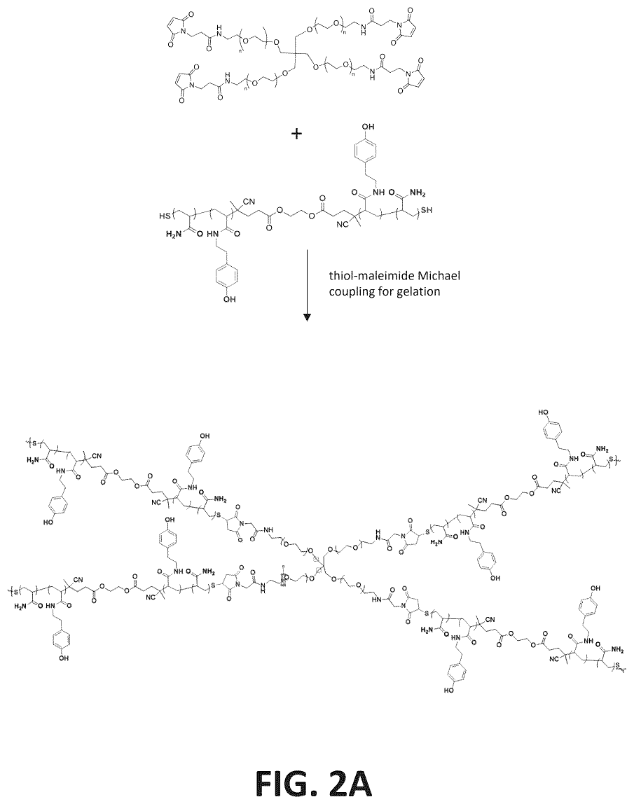

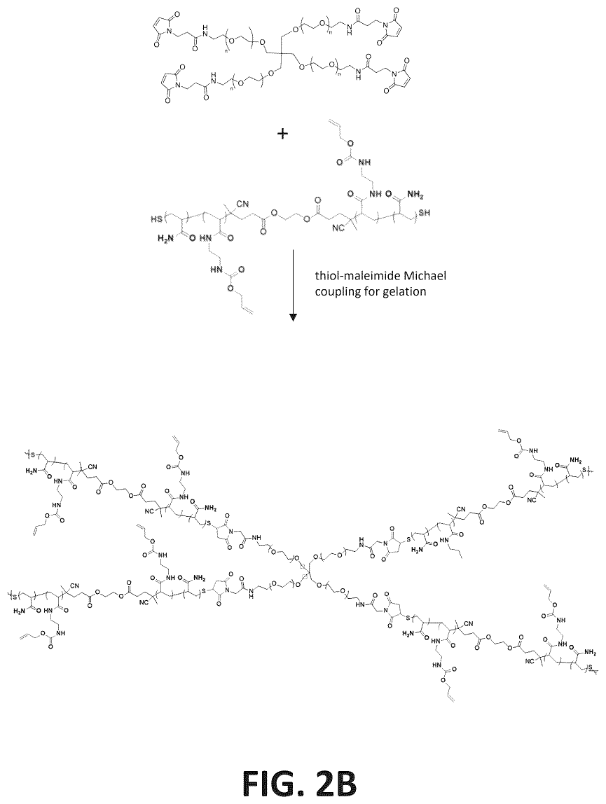

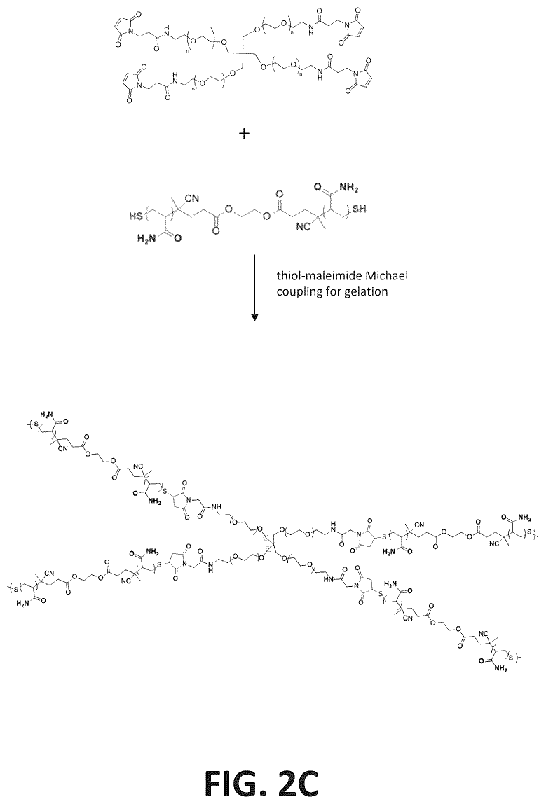

FIG. 2A, FIG. 2B, and FIG. 2C illustrate examples of a method of forming a matrix embedding the biological sample, wherein the matrix is the product of a polymerization reaction between the R^{A} of the multi-arm monomer or polymer and R^{B} of the second monomer or polymer. As illustrated in FIG. 2A, FIG. 2B, and FIG. 2C, in some embodiments the multi-arm monomer or polymer is a polymer comprising maleimide as illustrated in FIG. 2A, FIG. 2B, and FIG. 2C, and the second monomer or polymer is any of the polymers comprising thiol as illustrated.

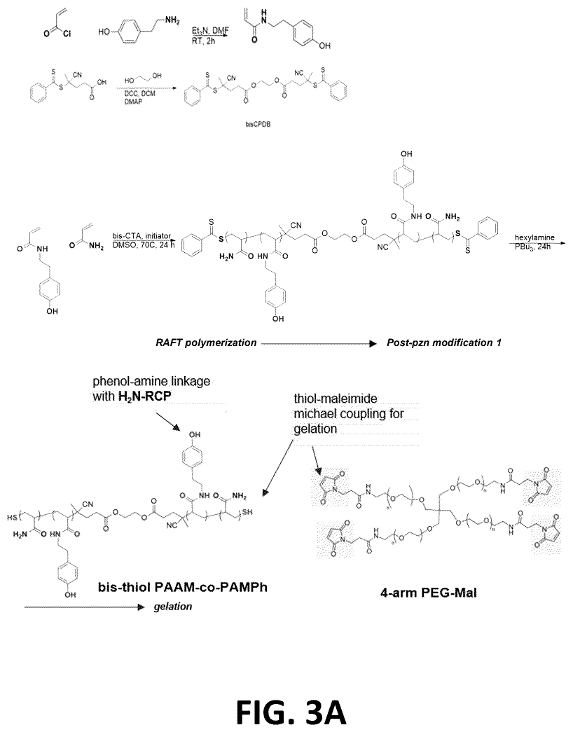

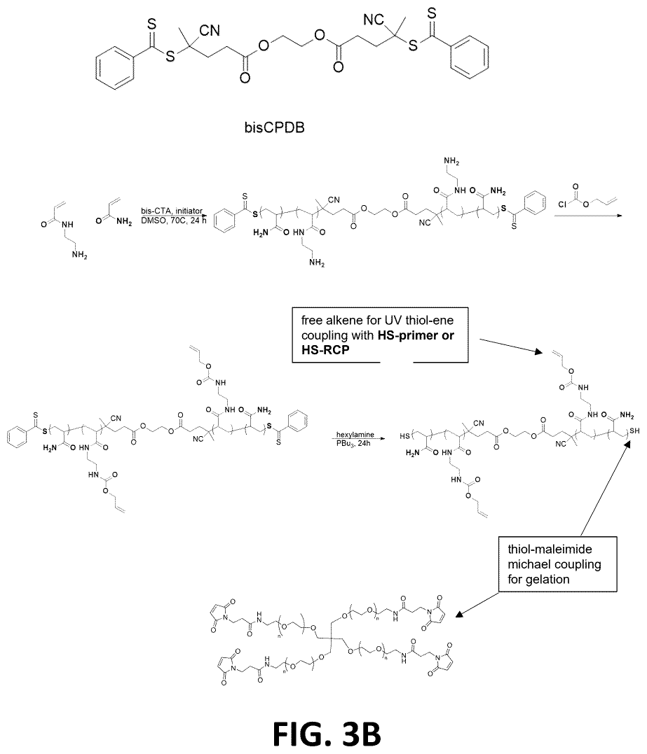

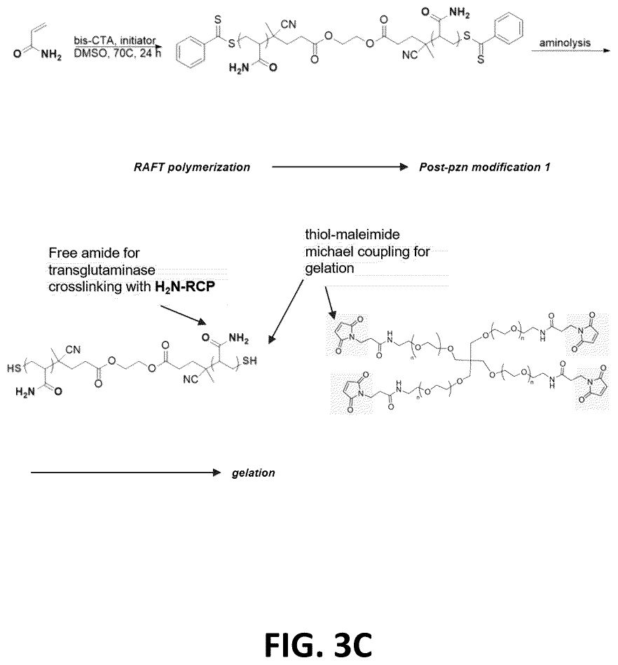

FIG. 3A illustrates an example of a method of synthesizing a second monomer or polymer. As shown in FIG. 3A, the backbone with pendant phenol groups is first formed and then the end functional groups are modified to comprise thiol. In some embodiments, the thiol is further cross-linked with the multi-arm monomer or polymer to form the matrix.

FIG. 3B illustrates another example of a method of synthesizing a second monomer or polymer. As shown in FIG. 3B, the backbone with pendant tethering moiety is first formed and then the end functional groups are modified to comprise thiol. In some embodiments, the thiol is further cross-linked with the multi-arm monomer or polymer to form the matrix.

FIG. 3C illustrates another example of a method of synthesizing a second monomer or polymer. As shown in FIG. 3C, the backbone with pendant tethering moiety is first formed and then the end functional groups are modified to comprise thiol. In some embodiments, the thiol is further cross-linked with the multi-arm monomer or polymer to form the matrix.

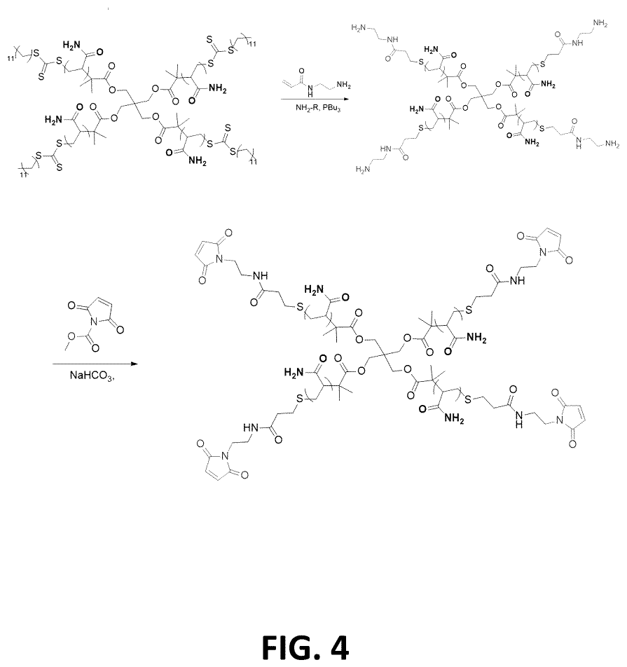

FIG. 4 illustrates an example of a method of synthesizing a multi-arm monomer or polymer. As shown in FIG. 4, the backbone of the multi-arm monomer or polymer is first formed and then the end functional groups are modified to comprise maleimide. In some embodiments, the maleimide is further cross-linked with the second monomer or polymer to form the matrix.

FIG. 5 illustrates an example of a method of forming a matrix embedding the biological sample, wherein the matrix is the product of a polymerization reaction between the R^{A} of the multi-arm monomer or polymer and R^{B} of the second monomer or polymer. As illustrated in FIG. 5, the multi-arm monomer or polymer is a 4-arm polymer comprising maleimide and the second monomer or polymer is a 4-arm polymer comprising thiol.

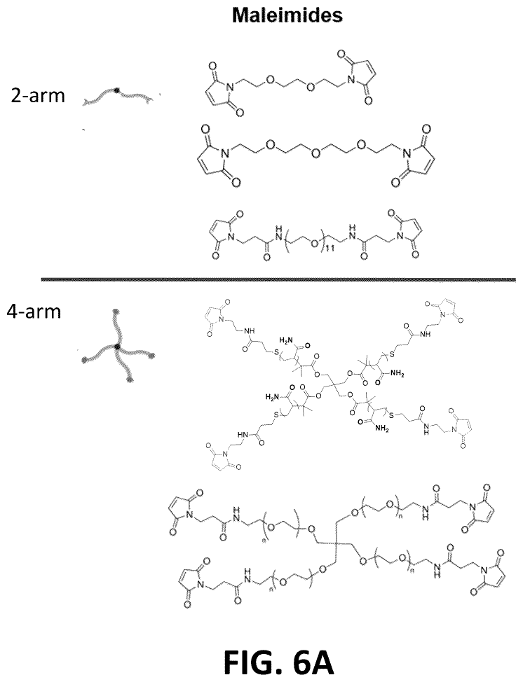

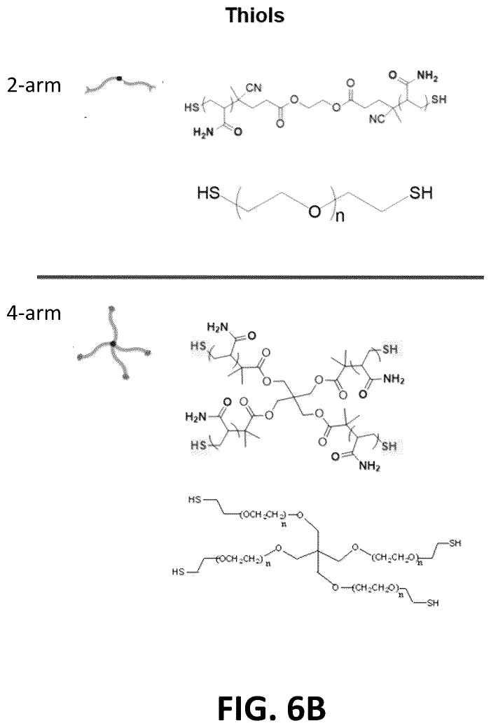

FIG. 6 illustrates examples of multi-arm monomers or polymers and second monomers or polymers for use, in some embodiments, with the methods described herein.

In some embodiments, the method further comprises chemically modifying a substrate. In some embodiments, the biological sample is on a chemically modified substrate. In some embodiments, the chemically modified substrate is capable of immobilizing the multi-arm monomer or polymer, the second monomer or polymer, or both.

In some embodiments, the chemically modified substrate is capable of forming covalent bond with R^{A} of the multi-arm monomer or polymer, R^{B} of the second monomer or polymer, or R^{T} of the second monomer or polymer. In some embodiments, the chemically modified substrate comprises an NHS ester, and at least one of R^{A}, R^{B}, or R^{T} comprises-NH₂. In some embodiments, the chemically modified substrate comprises an acrylamide, and at least one of R^{A}, R^{B}, or R^{T} comprises an acrylamide.

In some embodiments, the chemically modified substrate is capable of forming covalent bond with a linking reagent, and the linking reagent is capable of forming covalent bond with R^{A} of the multi-arm monomer or polymer, R^{B} of the second monomer or polymer, or R^{T} of the second monomer or polymer. In some embodiments, the chemically modified substrate comprises an NHS ester, and wherein the linking reagent comprises —NH₂ and a moiety capable of forming covalent bond with R^{A} of the multi-arm monomer or polymer, R^{B} of the second monomer or polymer, or R^{T} of the second monomer or polymer. In some embodiments, the chemically modified substrate comprises an acrylamide, and the linking reagent comprises an acrylamide and a moiety capable of forming covalent bond with R^{A} of the multi-arm monomer or polymer, R^{B} of the second monomer or polymer, or R^{T} of the second monomer or polymer.

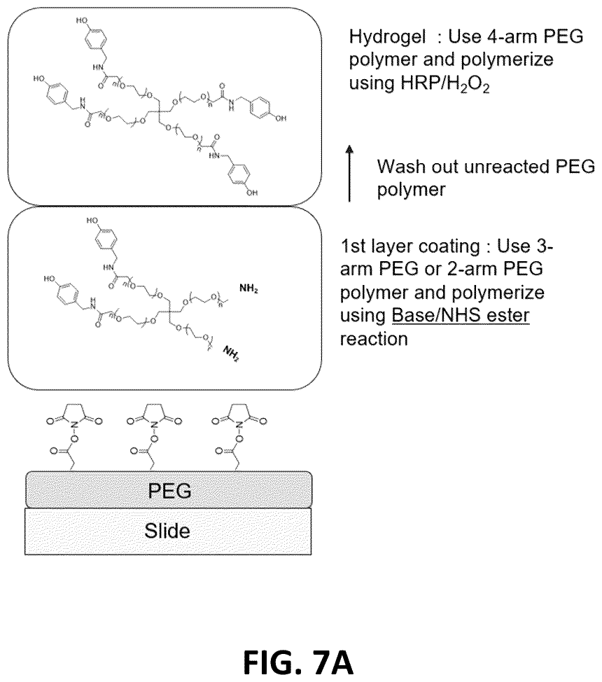

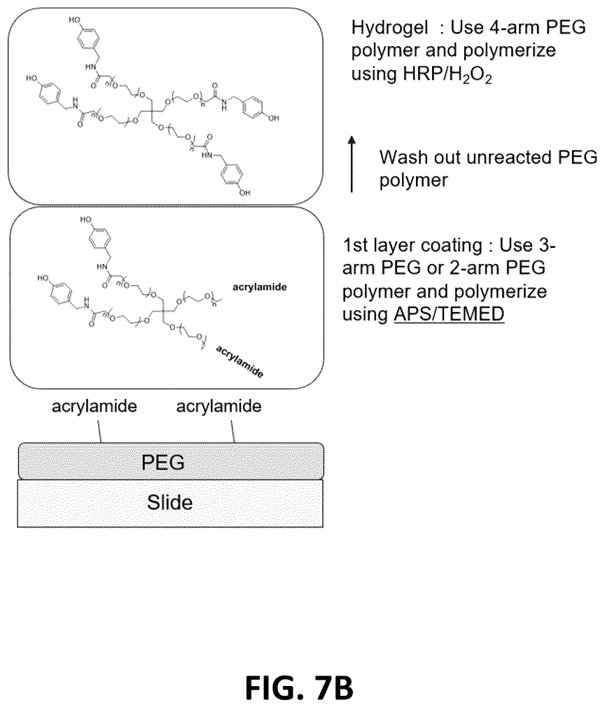

FIG. 7A and FIG. 7B illustrate examples of methods of modifying the substrate and further linking the multi-arm monomer or polymer onto the substrate. As illustrated in FIG. 7A, a linking reagent is used to link the multi-arm monomer or polymer onto the substrate, wherein the linking reagent comprises two amine groups and two phenol groups. In some embodiments, the amine groups react with the NHS ester groups on the substrate and the phenol groups further reacts with the multi-arm monomer or polymer. As illustrated in FIG. 7B, a linking reagent is used to link the multi-arm monomer or polymer onto the substrate, wherein the linking reagent comprises two acrylamide groups and two phenol groups. In some embodiments, the amine groups react with the acrylamide groups on the substrate and the phenol groups further react with the multi-arm monomer or polymer.

In one aspect, provided herein is a method for processing a biological sample, the method comprising: a) contacting the biological sample with a matrix-forming composition comprising a multi-arm monomer or polymer comprising a plurality of arms converging at a central branching point, each arm comprising a functional group R^{A} to form a complex of the biological sample and the multi-arm monomer or polymer; a′) contacting the complex with a second monomer or polymer comprising at least two functional groups R^{B} and a pendant tethering moiety R^{T}; b) forming a matrix embedding the biological sample, wherein the matrix is the product of a polymerization reaction between R^{A} of the multi-arm monomer or polymer and R^{B} of the second monomer or polymer; and c) attaching the tethering moiety R^{T} to an attachment moiety that is attached to an analyte, labeling agent, or nucleic acid amplification product, or any combination thereof in the biological sample, thereby attaching the analyte, labeling agent, or nucleic acid amplification product, or any combination thereof to the matrix.

In some embodiments, the biological sample is a fixed and/or permeabilized biological sample. In some embodiments, the biological sample is a tissue sample. In some embodiments, the biological sample is a frozen tissue sample or a fresh tissue sample. In some embodiments, the tissue sample is a tissue slice between about 1 μm and about 50 μm in thickness, optionally wherein the tissue slice is between about 5 μm and about 35 μm in thickness.

In some embodiments of the foregoing, the multi-arm monomer or polymer is any of the multi-arm monomers or polymers detailed herein. In some embodiments, the second monomer or polymer is any of the second monomers or polymers detailed herein.

In some aspects, the matrix is sufficiently optically transparent or otherwise has optical properties suitable for three dimensional imaging for high throughput information readout, such as for detection of probe or probe set (i.e., a detectable probe).

In some aspects, the matrix is porous thereby allowing the introduction of reagents into the matrix at the site of a RNA molecule comprising a free 3′ end. Porosity can result from polymerization and/or crosslinking of molecules used to make the matrix material. The diffusion property within the gel matrix is largely a function of the pore size. The molecular sieve size is chosen to allow for rapid diffusion of enzymes, oligonucleotides, formamide and other buffers used for amplification and sequencing (>50-nm). The molecular sieve size is also chosen so that large DNA or RNA amplicons do not readily diffuse within the matrix (<500-nm). The porosity is controlled by changing the cross-linking density, the chain lengths and the percentage of co-polymerized branching monomers. Additional control over the molecular sieve size and density of the matrix is achieved by adding additional cross-linkers such as functionalized polyethylene glycols. In some embodiments, the reagents introduced into the matrix include any of the reagents provided herein. In some embodiments, the reagents comprise a probe or probe set (e.g., detectable probe), amplification reagents (e.g., polymerase), and/or primers.

In some embodiments, the biological sample is reversibly cross-linked prior to or during an in situ assay. In some aspects, the analytes, polynucleotides and/or amplification product (e.g., amplicon) of an analyte or a probe bound thereto is/are anchored to the matrix. In some embodiments, one or more of the polynucleotide probe(s) and/or amplification product (e.g., amplicon) thereof is/are modified to contain a functional group (attachment moiety) that can be used as an anchoring site to attach the polynucleotide probe, primer, and/or amplification product to the tethering moiety (R^{T}) of the matrix. In some embodiments, a modified probe comprising oligo dT is used to bind to mRNA molecules of interest, followed by reversible or irreversible crosslinking of the mRNA molecules. In some embodiments, the modified probe comprising oligo dT is modified with an attachment moiety that can be attached to the tethering moiety (R^{T}) of the matrix. In some embodiments, the attachment moiety reacts with R^{T} with a reaction chemistry that is orthogonal to the gelation chemistry used for attachment of R^{A} to R^{B}.

In some embodiments, the biological sample is immobilized in the matrix via cross-linking of the multi-arm monomer or polymer and the second monomer or polymer that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, the matrix includes hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof. In some embodiments, the matrix includes hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), and combinations thereof.

In some embodiments, the matrix forms the substrate. In some embodiments, the substrate includes a matrix and one or more second materials. In some embodiments, the matrix is placed on top of one or more second materials. For example, the matrix can be pre-formed and then placed on top of, underneath, or in any other configuration with one or more second materials. In some embodiments, matrix formation occurs after contacting one or more second materials during formation of the substrate. In some embodiments, matrix formation occurs within a structure (e.g., wells, ridges, projections, and/or markings) located on a substrate.

In some embodiments, matrix formation on a substrate occurs before, contemporaneously with, or after probes are provided to the sample. For example, matrix formation can be performed on the substrate already containing the probes. In some embodiments, matrix formation occurs before a rolling circle amplification product is generated in the biological sample, and the rolling circle amplification product comprises an attachment moiety that is anchored to the tethering moiety R^{T} after matrix formation (e.g., using an orthogonal chemistry from the matrix formation chemistry). In some embodiments, the biological sample is cleared after attaching the attachment moiety to the tethering moiety.

In some embodiments, matrix formation occurs within a biological sample. In some embodiments, a biological sample (e.g., tissue section) is embedded in the matrix. In some embodiments, the matrix subunits are infused into the biological sample, and polymerization of the matrix is initiated by an external or internal stimulus. In some embodiments, polymerization of the matrix is initiated by an external or internal stimulus that is different from a stimulus used to promote attachment of the attachment moiety to the tethering moiety.

In embodiments in which a matrix is formed within a biological sample, functionalization chemistry is used. In some embodiments, functionalization chemistry includes hydrogel-tissue chemistry (HTC). Any hydrogel-tissue backbone (e.g., synthetic or native) suitable for HTC is used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible. In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, and dNTPs used to amplify the nucleic acid. In some embodiments, one or more other enzymes are used, including without limitation: RNA polymerase, ligase, proteinase K, and DNAse. In some embodiments, additional reagents include reverse transcriptase enzymes, including enzymes with terminal transferase activity, primers, and oligonucleotides. In some embodiments, optical labels (e.g., detectable labeled probes) are added to the hydrogel subunits before, contemporaneously with, and/or after polymerization.

### III. Samples, Analytes, and Detection

### A. Samples Preparation and Staining

Provided herein are methods for processing and/or analyzing biological samples. A sample disclosed herein can be or be derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. In some embodiments, a biological sample is obtained from non-mammalian organisms (e.g., a plant, an insect, an arachnid, a nematode, a fungus, or an amphibian). In some embodiments, a biological sample is obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). In some embodiments, the biological sample includes nucleic acids (such as DNA or RNA), proteins/polypeptides, carbohydrates, and/or lipids. In some embodiments, the biological sample is obtained as a tissue sample, such as a tissue section, biopsy, a core biopsy, needle aspirate, or fine needle aspirate. In some embodiments, the sample is a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample comprises cells which are deposited on a surface, cells from a portion of a cell block or cells from a portion of a cell pellet.

Biological samples can be derived from a homogeneous culture or population of the subjects or organisms mentioned herein or alternatively from a collection of several different organisms.

In some embodiments, the biological sample comprises one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix (e.g., as described in Section II). In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein is any support that is insoluble in aqueous liquid and which allows for positioning of biological samples, analytes, features, and/or reagents (e.g., probes) on the support. In some embodiments, a biological sample is attached to a substrate. Attachment of the biological sample can be irreversible or reversible, depending upon the nature of the sample and subsequent steps in the analytical method. In certain embodiments, the sample is attached to the substrate reversibly by applying a suitable polymer coating to the substrate, and contacting the sample to the polymer coating. In some embodiments, the sample is then detached from the substrate, e.g., using an organic solvent that at least partially dissolves the polymer coating. Hydrogels are examples of polymers that are suitable for this purpose.

In some embodiments, the substrate is coated or functionalized with one or more substances to facilitate attachment of the sample to the substrate. Suitable substances that can be used to coat or functionalize the substrate include, but are not limited to, lectins, poly-lysine, antibodies, and polysaccharides.

In some embodiments, the biological sample comprises an exogenous or endogenous molecule. In some embodiments, the endogenous molecule comprises a RNA. In some embodiments, the biological sample comprises an analyte. In some embodiments, the analyte comprises a RNA. In some embodiments, the biological sample comprises a RNA.

In some embodiments, the biological sample comprises cells or cellular components. In some embodiments, the biological sample comprises a tissue.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Preparation

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analyzed. In some embodiments, the tissue slice is between about 1-50 μm in thickness.

In some embodiments, multiple sections are obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analyzed successively to obtain three-dimensional information about the biological sample.

In some embodiments, the biological sample (e.g., a tissue section as described above) is prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

In some embodiments, the biological sample is a frozen tissue sample. In other embodiments, the biological sample is a fresh tissue sample. In some embodiments, the sample is a fresh frozen tissue sample (FF). In some embodiments, the sample is a tissue section of a tissue block.

In some embodiments, the biological sample is prepared using formalin-fixation and paraffin-embedding (FFPE), which are established methods. In some embodiments, FFPE is performed prior to embedding the sample in a matrix. In some embodiments, cell suspensions and other non-tissue samples are prepared using formalin-fixation and paraffin-embedding. Following fixation of the sample and embedding in a paraffin or resin block, the sample can be sectioned as described above. Prior to analysis (e.g., prior to matrix-embedding or introduction of a matrix forming material), the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes). In some embodiments, the biological sample (e.g., FFPE sample) is permeable after deparaffinization. In some embodiments, processing of the biological sample, such as de-waxing, allows the biological sample to become permeabilized.

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, the methods provided herein comprise one or more post-fixing (also referred to as postfixation) steps. In some embodiments, one or more post-fixing step is performed after contacting a sample with a polynucleotide disclosed herein, e.g., a probe. In some embodiments, one or more post-fixing step is performed after a hybridization complex comprising the nucleic acid molecule and a target is formed in a sample. In some embodiments, one or more post-fixing step is performed prior to a ligation reaction disclosed herein.

In some embodiments, a method disclosed herein comprises de-crosslinking the reversibly cross-linked biological sample. The de-crosslinking does not need to be complete.

In some embodiments, a biological sample is permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample is incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample is permeabilized by any suitable methods. For example, one or more lysis reagents can be added to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

(ii) Clearing and/or Isometric Expansion

In some embodiments, a biological sample embedded in the three-dimensional polymerized matrix (e.g., hydrogel) is cleared using any suitable method (e.g., by contacting the biological sample with a clearing agent). A clearing agent can be any suitable agent for clearing a biological sample. For example, biological samples embedded in the three-dimensional polymerized matrix can be cleared with a detergent, a lipase and/or a protease. In some embodiments, the detergent and lipase removes fatty molecules. In some embodiments, the detergent comprises an ionic detergent or a nonionic detergent. In some embodiments, the detergent comprises a non-ionic surfactant or an anionic surfactant. In some embodiments, the detergent comprises SDS, tergitol, NP-40, saponin, p-(1,1,3,3-tetramethylbutyl)-phenyl ether (also known as Triton X-100™, octyl phenol ethoxylate, polyoxyethylene octyl phenyl ether, 4-octylphenol polyethoxylate, t-octylphenoxypolyethoxyethanol, and octoxynol-9), polysorbate 20 (also known as Tween-20™ polyoxyethylene (20) sorbitan monolaurate, or PEG(20) sorbitan monolaurate), or any combinations thereof. In some embodiments, the lipase comprises a pancreatic, hepatic and/or lysosomal lipase, or any combinations thereof. In some embodiments, the lipase comprises sphingomyelinase or esterase, or a combination thereof. In some embodiments, the protease targets extracellular matrix, fibronectin, collagen and/or elastin. In some embodiments, the protease comprises proteinase K, pepsin, collagenase, trypsin, dispase, thermolysin, or alpha-chymotrypsin, or any combinations thereof. In some embodiments, the protease comprises proteinase K, pepsin, collagenase, trypsin, dispase, thermolysin, or alpha-chymotrypsin, or any combinations thereof. In some embodiments, the protease comprises Liberase™, (Collagenase I, Collagenase II and thermolysin). In some embodiments, proteins in the biological sample are crosslinked to the matrix before treatment with a protease (e.g., proteinase K). In other embodiments, electrophoretic tissue clearing methods are used to remove biological macromolecules from the hydrogel-embedded sample. In some embodiments, the detergent can remove protein (e.g., ribosome) from the tethered RNA. In some embodiments, a hydrogel-embedded sample is stored before or after clearing of hydrogel, in a medium (e.g., a mounting medium, methylcellulose, or other semi-solid mediums).

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a biological sample embedded in a matrix (e.g., a hydrogel) is isometrically expanded. Isometric expansion methods that can be used include hydration, a preparative step in expansion microscopy, as described in, e.g., Chen et al., Science 347(6221): 543-548, 2015 and U.S. Pat. No. 10,059,990, which are herein incorporated by reference in their entireties. Isometric expansion of the sample can increase the spatial resolution of the subsequent analysis of the sample. The increased resolution in spatial profiling can be determined by comparison of an isometrically expanded sample with a sample that has not been isometrically expanded. In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(iii) Staining and Immunohistochemistry (IHC)

To facilitate visualization, biological samples can be stained using a wide variety of stains and staining techniques. In some embodiments, for example, a sample is stained using any number of stains and/or immunohistochemical reagents. One or more staining steps may be performed to prepare or process a biological sample for an assay described herein or may be performed during and/or after an assay. In some embodiments, the sample is contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample are segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but not limited to, acridine orange, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, a biological sample is destained. Any suitable methods of destaining or discoloring a biological sample may be utilized, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65 (8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

In some embodiments, the embedded biological sample is stained.

### B. Analytes

In some aspects, provided herein are new and improved methods and compositions for embedding a biological sample in a matrix and detecting one or more analytes present in the biological sample embedded in a matrix. A biological sample may comprise one or a plurality of analytes of interest. Methods for performing multiplexed assays to analyze two or more different analytes in a single biological sample are provided. The methods disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte).

Analytes of particular interest may include nucleic acid molecules, such as RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-RNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as RNA, e.g. interactions between proteins and nucleic acids, e.g. regulatory factors, such as transcription factors, and RNA.

### (i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., by immobilizing or tethering any fragmented RNAs to an endogenous molecule in the biological sample or an exogenous molecule delivered to the biological sample as described in Section II and using a nucleic acid probe or probe set that directly or indirectly hybridizes to the immobilized or tethered nucleic acid analyte).

Examples of nucleic acid analytes include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. In some embodiments, the RNA is small (e.g., less than 200 nucleic acid bases in length). In some embodiments, the RNA is large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA). In some embodiments described herein, an analyte is a fragmented RNA.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature (e.g., region) of the substrate.

### (ii) Labelling Agents

In some embodiments, provided herein are methods and compositions for analyzing endogenous analytes (e.g., RNA) in a sample and other analytes using one or more labelling agents. In some embodiments, the analyte is a nucleic acid analyte (e.g., RNA), and the labelling agent is a nucleic acid probe that binds directly or indirectly to the nucleic acid analyte or to a product of the nucleic acid analyte, such as a cDNA. In some embodiments, the labelling agent is functionalized with an attachment moiety. In some embodiments, the attachment moiety is a 5′ acrydite.

In some aspects, the method is compatible with protein analysis (e.g., using a labelling agent). In some embodiments, an analyte labelling agent may include an agent that interacts with an analyte (e.g., an endogenous analyte in a sample). In some embodiments, the labelling agents comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent is further contacted with a probe (e.g., a single-stranded probe sequence), that hybridizes to a reporter oligonucleotide of the labelling agent, in order to identify the analyte associated with the labelling agent. In some embodiments, the analyte labelling agent comprises an analyte binding moiety and a labelling agent nucleic acid barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein. In some embodiments, the labelling agent is functionalized with an attachment moiety.

In some embodiments, the method comprises one or more post-fixing (also referred to as post-fixation) steps after contacting the sample with one or more labelling agents.

In some embodiments, an analyte binding moiety may include any molecule or moiety capable of binding to an analyte (e.g., a biological analyte, e.g., a macromolecular constituent). A binding moiety may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The non-nucleic acid analyte labelling agents can include (e.g., are attached to) a reporter oligonucleotide that is indicative of the cell surface feature to which the binding group binds. In some embodiments, the reporter oligonucleotide is functionalized with an attachment moiety.

For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labelling agents are the same. In some embodiments in which the plurality of analytes includes a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labelling agents may be achieved through any of a variety of direct or indirect, covalent or non-covalent associations or attachments. In some embodiments, the oligonucleotide attached to a labelling agent comprises a sequence that can serve as a primer and can be used as a reporter (e.g., a barcode). In some embodiments, the oligonucleotide attached to a labelling agent comprises both a reporter sequence (e.g., a barcode) and a sequence serving as a primer. In some embodiments, the oligonucleotide attached to a labelling agent comprises a reporter sequence (e.g., a barcode), a sequence serving as a primer, and an attachment moiety For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31 (2): 708-715, the content of which is herein incorporated by reference in its entirety. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the labelling agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide or primer may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence or a unique molecular identifier (UMI) sequence.

In some cases, the labelling agent comprises a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

### C. Detection and Analysis

In some aspects, the provided methods involve analyzing the tethered or immobilized analytes, probes, or amplification products, e.g., by detecting or determining, one or more sequences present in probes or probe sets or products thereof (e.g., rolling circle amplification products thereof). In some embodiments, the detecting is performed at one or more locations in a biological sample. In some embodiments, the locations are the locations of tethered or immobilized RNA transcripts in the biological sample. In some embodiments, the locations are the locations at which probes or probe sets hybridize to the RNA transcripts in the biological sample, and are optionally ligated and amplified by rolling circle amplification.

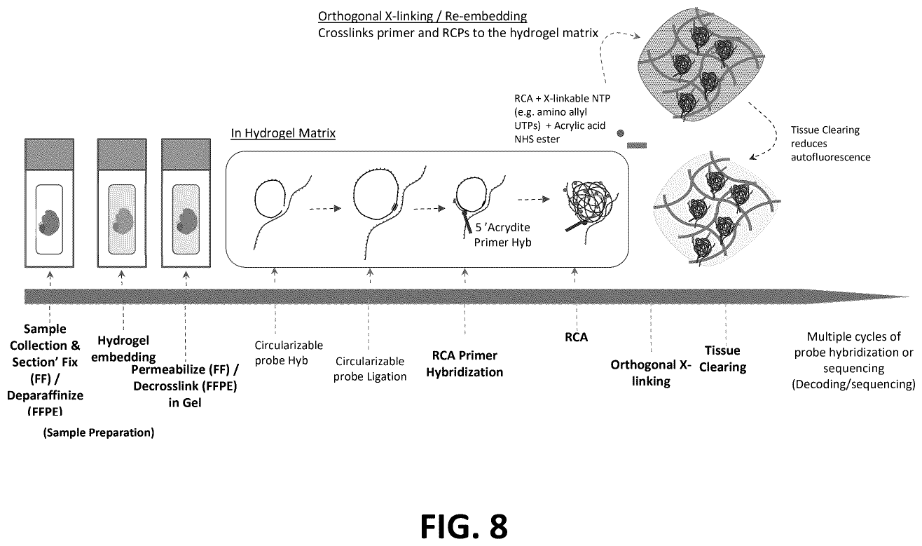

In some embodiments, the method provided herein comprises contacting the biological sample with a probe or probe set that binds directly or indirectly to the RNA (e.g., as shown in FIG. 8). In some embodiments, the probe or probe set is a detectable probe. In some embodiments, the probe or probe set comprises one or more probes modified with an attachment moiety that can be attached to R^{T} as described in Section II. In some embodiments, the probe or probe set is a circular or circularizable probe or probe set. In some embodiments, the method comprises circularizing the circularizable probe or probe set using the RNA or a product thereof as a template. In some embodiments, the method comprises generating an RCA product using the circular or circularizable probe as a template. In some embodiments, the RCA product comprises an attachment moiety that can be attached to R^{T} as described in Section II. In certain embodiments, the attachment moiety is incorporated into the RCA product by contacting the biological sample with a primer comprising the attachment moiety, wherein the primer hybridizes to the circular or circularizable probe and is used to prime the RCA. In certain embodiments, the attachment moiety is incorporated into the RCA product by contacting the biological sample with one or more modified nucleotides comprising the attachment moiety, wherein the one or more modified nucleotides are incorporated into the RCA product. In some embodiments, the method further comprises detecting a signal associated with a fluorescently labeled probe that directly or indirectly binds to the RCA product.

In some embodiments, the probe or probe set comprises a barcode sequence. In some embodiments, the method comprises detecting the barcode sequence or a complement thereof in the probe or probe set or in a product of the probe or probe set. In some embodiments, the probe or probe set comprises an intermediate probe and a detection oligonucleotide. In some embodiments, the detecting comprises a plurality of repeated cycles of hybridization and removal of probes (e.g., detectably labeled probes, or intermediate probes that bind to detectably labeled probes) to the primary probe or probe set hybridized to the target nucleic acid, or to a rolling circle amplification product generated from the probe or probe set hybridized to the target nucleic acid.

In some embodiments, the method comprises contacting the biological sample with a circular or circularizable probe, wherein the circular or circularizable probe binds the fragmented RNA and generates a rolling circle amplification (RCA) product.

In some embodiments, the method comprises imaging the biological sample to detect a signal associated a probe or probe set (e.g., a circular or circularizable probe or probe set) that binds directly or indirectly to the RNA. In some embodiments, the method comprises imaging the biological sample to detect the RCA product. In some embodiments, imaging comprises detecting a signal associated the probe or probe set or the RCA product, optionally with a fluorescently labeled probe that directly or indirectly binds to the RCA product.

### (a) Hybridization, Ligation, and Amplification

In some embodiments, the method comprises contacting the biological sample with a probe or probe set that binds (e.g., hybridizes) directly or indirectly to the RNA. For purposes of hybridization, two nucleic acid sequences are “substantially complementary” if at least 60% (e.g., at least 70%, at least 80%, or at least 90%) of their individual bases are complementary to one another. Various probes and probe sets can be hybridized to an endogenous analyte (e.g., RNA) and/or a labelling agent.

In some aspects, “binding” as used herein refers to the coupling between two or more nucleic acids, e.g., oligonucleotides and/or polynucleotides. In some embodiments, the binding is indirect binding. In some embodiments, the binding is direct (e.g., binding comprising direct hybridization of nucleic acid sequences). The nature of the binding may vary. In some instances, a first nucleic acid sequence directly binds to a second nucleic acid sequence via hybridization of complementary sequences. In some instances, a first nucleic acid sequence indirectly binds to a second nucleic acid sequence via one or more intermediate nucleic acids. For example, an intermediate nucleic acid comprises a first region that binds to the first nucleic acid sequence and has a second region for binding to the second nucleic acid sequence, thereby forming a complex comprising the first and second nucleic acid sequences.

In some embodiments, the method comprises generating a ligation product with a probe or probe set that hybridizes directly or indirectly to the RNA.

In some embodiments, provided herein is a probe or probe set capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 20190055594, which is hereby incorporated by reference in its entirety. In some embodiments, provided herein is a multiplexed proximity ligation assay. See, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe is indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps”. In some embodiments, said ends are not ligated directly to each other, but instead occurs either via the intermediacy of one or more intervening (so-called “gap” or “gap-filling” (oligo) nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo) nucleotide(s) which are complementary to a splint, circularizable probe (e.g., padlock probe), or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo) nucleotide, such that the gap (oligo) nucleotide becomes incorporated into the resulting polynucleotide. In some embodiments, the ligation herein is preceded by gap filling. In other embodiments, the ligation herein does not require gap filling.

In some embodiments, the method comprises detecting a product that includes a molecule or a complex generated in a series of reactions, e.g., hybridization, ligation, extension, replication, transcription/reverse transcription, and/or amplification (e.g., rolling circle amplification), in any suitable combination. In some embodiments, the RCA is performed using an exogenous primer comprising an attachment moiety. In some embodiments, the RCA is performed using the target nucleic acid (e.g., RNA) as a primer. In some embodiments, the RNA is attached to the matrix.

In some embodiments, amplification (e.g., rolling circle amplification) is primed by the target RNA. In some embodiments, the target RNA is cleaved by an enzyme (e.g., RNase H). In some embodiments, the target RNA is cleaved at a position downstream of the probe binding site(s). In some aspects, the methods disclosed herein allow targeting of RNase H activity to a particular region in a target RNA that is adjacent to or overlapping with a target sequence for the nucleic acid probe or probe set. For example, a nucleic acid oligonucleotide is designed to hybridize to a complementary oligonucleotide hybridization region in the target RNA. In some embodiments, a nucleic acid oligonucleotide is used to provide a DNA-RNA duplex for RNase H cleavage of the target RNA in the DNA-RNA duplex. In some embodiments, the oligonucleotide binds to the target RNA at a position that overlaps with the target sequence of the probe or probe set by about 1 to about 20 nucleotides or by about 8 to about 10 nucleotides. In some embodiments, the cleaved target RNA itself is then used to prime RCA of the circularized probe generated from the probe or probe set (e.g., target-primed RCA). In some cases, a plurality of nucleic acid oligonucleotides are used to perform target-primed RCA for a plurality of different target RNAs.

In some embodiments, the biological sample is contacted with the oligonucleotide and with the RNase H simultaneously or sequentially (in either order) before contacting the sample with the probe or probe set. In some embodiments, the probe or probe set hybridizes to the cleaved target RNA, and the cleaved target RNA is used to prime RCA. In any of the embodiments herein, the RNase H comprises an RNase H1 and/or an RNAse H2. In some embodiments, RNase inactivating agents or inhibitors are added to the sample after cleaving the target RNA.

In some embodiments, the RCA incorporates one or more nucleotides modified with an attachment moiety. For example, in certain embodiments the RCA is performed using amino allyl UTPs to replace some of all dTTPs in the nucleotide reaction mixture contacted with the biological sample for the RCA.

In some aspects, the polynucleotides and/or amplification product (e.g., amplicon) are anchored to the polymer matrix. For example, the polymer matrix can be any of the matrices produced according to the present disclosure. In some examples, the scaffold also contains modifications or functional groups that can react with or incorporate the modifications or functional groups of the probe set or amplification product. In some examples, the scaffold comprises oligonucleotides, polymers or chemical groups, to provide a matrix and/or support structures.

In some embodiments, the amplification products are immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot.

In some embodiments, a method disclosed herein may also comprise one or more signal amplification components. In some embodiments, the present disclosure relates to the detection of nucleic acids sequences in situ using probe hybridization and generation of amplified signals associated with the probes, wherein background signal is reduced and sensitivity is increased. In some embodiments, an analyte disclosed herein is detected using a method that comprises signal amplification. Examples of signal amplification methods include targeted deposition of detectable reactive molecules around the site of probe hybridization, targeted assembly of branched structures (e.g., bDNA or branched assay using locked nucleic acid (LNA)), programmed in situ growth of concatemers by enzymatic rolling circle amplification (RCA) (e.g., as described in US 2019/0055594 incorporated herein by reference), hybridization chain reaction, assembly of topologically catenated DNA structures using serial rounds of chemical ligation (clampFISH), signal amplification via hairpin-mediated concatemerization (e.g., as described in US 2020/0362398 incorporated herein by reference), e.g., primer exchange reactions such as signal amplification by exchange reaction (SABER) or SABER with DNA-Exchange (Exchange-SABER). In some embodiments, a non-enzymatic signal amplification method may be used.

The detectable reactive molecules may comprise tyramide, such as used in tyramide signal amplification (TSA) or multiplexed catalyzed reporter deposition (CARD)-FISH. In some embodiments, the detectable reactive molecule may be releasable and/or cleavable from a detectable label such as a fluorophore. In some embodiments, a method disclosed herein comprises multiplexed analysis of a biological sample comprising consecutive cycles of probe hybridization, fluorescence imaging, and signal removal, where the signal removal comprises removing the fluorophore from a fluorophore-labeled reactive molecule (e.g., tyramide). Exemplary detectable reactive reagents and methods are described in U.S. Pat. No. 6,828,109, US 2019/0376956, US 2019/0376956, WO 2020/102094, WO 2020/163397, and WO 2021/067475, all of which are incorporated herein by reference in their entireties.

The detectable label can be directly detectable by itself (e.g., radioisotope labels or fluorescent labels) or, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some embodiments, the detectable label is bound to a feature or to a capture probe associated with a feature. For example, detectably labelled features can include a fluorescent, a colorimetric, or a chemiluminescent label attached to a bead (see, for example, Rajeswari et al., J. Microbiol Methods 139:22-28, 2017, and Forcucci et al., J. Biomed Opt. 10:105010, 2015, the entire contents of each of which are incorporated herein by reference).

In some embodiments, a plurality of detectable labels are attached to a probe, or composition to be detected. For example, detectable labels can be incorporated during nucleic acid polymerization or amplification (e.g., Cy5®-labelled nucleotides, such as Cy5®-dCTP). Any suitable detectable label can be used. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18 (5)), DIDS, Dil (DilC18 (3)), DiO (DiOC18 (3)), DiR (DilC18 (7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARFR-1 (high pH), SNARFR-1 (low pH), Sodium Green™, SpectrumAqua®, SpectrumGreen® #1, SpectrumGreen® #2, SpectrumOrange®, SpectrumRed®, SYTOR 11, SYTOR 13, SYTOR 17, SYTOR 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

As mentioned above, in some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. A number of compound families provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and -methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label. For example, small cluster metal ions, metals, or semiconductors may act as a mass code. In some examples, the metals can be selected from Groups 3-15 of the periodic table, e.g., Y, La, Ag, Au, Pt, Ni, Pd, Rh, Ir, Co, Cu, Bi, or a combination thereof.

In some embodiments, hybridization chain reaction (HCR) is used for in situ signal amplification and detection. HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target reporter oligonucleotide. HCR is described in detail in Dirks and Pierce, 2004, PNAS, 101 (43), 15275-15278 and in U.S. Pat. No. 7,632,641 (see also US 2006/00234261; Chemeris et al., 2008 Doklady Biochemistry and Biophysics, 419, 53-55; Niu et al., 2010, 46, 3089-3091; Choi et al., 2010, Nat. Biotechnol. 28 (11), 1208-1212; and Song et al., 2012, Analyst, 137, 1396-1401). HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be known as the “toehold region” (or “input domain”). The first HCR monomers each further comprise a sequence which is complementary to a sequence in the exposed toehold region of the second HCR monomers. This sequence of complementarity in the first HCR monomers may be known as the “interacting region” (or “output domain”). Similarly, the second HCR monomers each comprise an interacting region (output domain), e.g. a sequence which is complementary to the exposed toehold region (input domain) of the first HCR monomers. In the absence of the HCR initiator, these interacting regions are protected by the secondary structure (e.g. they are not exposed), and thus the hairpin monomers are stable or kinetically trapped (also referred to as “metastable”), and remain as monomers (e.g. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g. the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers could readily be conceived.

### (b) Detection and Analysis

A target sequence for a probe or probe set disclosed herein may be comprised in any analyte disclose herein, including an endogenous analyte (e.g., a probe or probe set that hybridizes directly or indirectly to the RNA), a labelling agent, or a product or derivative of an endogenous analyte and/or a labelling agent.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the two or more sub-barcodes are overlapping sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In any of the embodiments herein, barcodes (e.g., primary and/or secondary barcode sequences) are analyzed (e.g., detected or sequenced) using any suitable methods or techniques, including those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), in situ sequencing, hybridization-based in situ sequencing (HybISS), targeted in situ sequencing, fluorescent in situ sequencing (FISSEQ), sequencing by synthesis (SBS), sequencing by ligation (SBL), sequencing by hybridization (SBH), or spatially-resolved transcript amplicon readout mapping (STARmap). In any of the preceding embodiments, the methods provided herein can include analyzing the barcodes by sequential hybridization and detection with a plurality of labelled probes (e.g., detection oligos).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4N complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (45=1024), whereas 8 nucleotide barcodes can be used to identify up to 65,536 molecular species, a number greater than the total number of distinct genes in the human genome. In some embodiments, the barcode sequences rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and US 2021/0164039, which are hereby incorporated by reference in their entirety.

In some embodiments, detection of the barcode sequences is performed by sequential hybridization of probes to the barcode sequences or complements thereof and detecting complexes formed by the probes and barcode sequences or complements thereof. In some cases, each barcode sequence or complement thereof is assigned a sequence of signal codes that identifies the barcode sequence or complement thereof (e.g., a temporal signal signature or code that identifies the analyte), and detecting the barcode sequences or complements thereof comprises decoding the barcode sequences of complements thereof by detecting the corresponding sequences of signal codes detected from sequential hybridization, detection, and removal of sequential pools of intermediate probes and the universal pool of detectably labeled probes. In some cases, the sequences of signal codes can be fluorophore sequences assigned to the corresponding barcode sequences or complements thereof. In some embodiments, the detectably labeled probes are fluorescently labeled. In some embodiments, the barcode sequence or complement thereof is performed by sequential probe hybridization as described in US 2021/0340618, the content of which is herein incorporated by reference in its entirety.

In any of the embodiments herein, the detecting step comprises contacting the biological sample with one or more detectably labeled probes that directly or indirectly hybridize to the barcode sequences or complements thereof (e.g., in amplification products generated using the probes or probe sets), and dehybridizing the one or more detectably labeled probes. In any of the embodiments herein, the contacting and dehybridizing steps can be repeated with the one or more detectably labeled probes and/or one or more other detectably labeled probes that directly or indirectly hybridize to the barcode sequences or complements thereof. In some aspects, the method comprises sequential hybridization of detectably labeled probes to create a spatiotemporal signal signature or code that identifies the analyte. In some aspects, the methods involving enzymatic and chemical reactions described herein (e.g., provided in Section II) can preserve the sample and maintain the spatial localization of the analytes through the repeated steps of probe hybridization and removal during the detecting step.

In any of the embodiments herein, the detecting step can comprise contacting the biological sample with one or more first detectably labeled probes that directly hybridize to the plurality of probes or probe sets. In some instances, the detecting step comprises contacting the biological sample with one or more first detectably labeled probes that indirectly hybridize to the plurality of probes or probe sets. In any of the embodiments herein, the detecting step comprises contacting the biological sample with one or more first detectably labeled probes that directly or indirectly hybridize to the plurality of probes or probe sets.

In any of the embodiments herein, the detecting step comprises contacting the biological sample with one or more intermediate probes that directly or indirectly hybridize to the barcode sequences or complements thereof (e.g., of the plurality of probes or probe sets or rolling circle amplification product generated using the plurality of probes or probe sets), wherein the one or more intermediate probes are detectable using one or more detectably labeled probes.

In any of the embodiments herein, the detecting step further comprises removing the signal(s) of the one or more hybridized detectably labeled probes (e.g., by quenching, cleaving the label, and/or performing one or more washes). In any of the embodiments herein, the detecting further comprises dehybridizing the one or more intermediate probes and/or the one or more detectably labeled probes from the barcode sequences or complements thereof (e.g., of the plurality of probes or probe sets or rolling circle amplification product generated using the plurality of probes or probe sets). In any of the embodiments herein, the contacting and dehybridizing steps is repeated with the one or more intermediate probes, the one or more detectably labeled probes, one or more other intermediate probes, and/or one or more other detectably labeled probes.

In some embodiments, sequence detection is performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309:1728-1732, and in U.S. Pat. Nos. 5,599,675; 5,750,341; 6,969,488; 6,172,218; and 6,306,597, all of which are herein incorporated by reference in their entireties.

In some embodiments, all or a portion of the RCP (e.g., a barcode sequence in the RCP) is detected using a base-by-base sequencing method, e.g., SBS or SBB. In some embodiments, the biological sample is contacted with a sequencing primer and base-by-base sequencing using a cyclic series of nucleotide incorporation or binding, respectively, thereby generating extension products of the sequencing primer is performed followed by removing, cleaving, or blocking the extension products of the sequencing primer.

In some embodiments, sequencing is performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Example SBS methods comprise those described for example, but not limited to, US 2007/0166705, US 2006/0188901, U.S. Pat. No. 7,057,026, US 2006/0240439, US 2006/0281109, US 2011/0059865, US 2005/0100900, U.S. Pat. No. 9,217,178, US 2009/0118128, US 2012/0270305, US 2013/0260372, and US 2013/0079232, all of which are herein incorporated by reference in their entireties.

In some embodiments, sequencing is performed by sequencing-by-binding (SBB). Various aspects of SBB are described in U.S. Pat. No. 10,655,176 B2, the content of which is herein incorporated by reference in its entirety. In some embodiments, SBB comprises performing repetitive cycles of detecting a stabilized complex that forms at each position along the template nucleic acid to be sequenced (e.g. a ternary complex that includes the primed template nucleic acid, a polymerase, and a cognate nucleotide for the position), under conditions that prevent covalent incorporation of the cognate nucleotide into the primer, and then extending the primer to allow detection of the next position along the template nucleic acid. In the sequencing-by-binding approach, detection of the nucleotide at each position of the template occurs prior to extension of the primer to the next position. Generally, the methodology is used to distinguish the four different nucleotide types that can be present at positions along a nucleic acid template by uniquely labelling each type of ternary complex (i.e. different types of ternary complexes differing in the type of nucleotide it contains) or by separately delivering the reagents needed to form each type of ternary complex. In some instances, the labeling may comprise fluorescence labeling of, e.g., the cognate nucleotide or the polymerase that participate in the ternary complex.

In some embodiments, sequencing is performed by sequencing-by-avidity (SBA). Some aspects of SBA approaches are described in U.S. Pat. No. 10,768,173 B2, the content of which is herein incorporated by reference in its entirety. In some embodiments, SBA comprises detecting a multivalent binding complex formed between a fluorescently-labeled polymer-nucleotide conjugate, and a one or more primed target nucleic acid sequences (e.g., barcode sequences). Fluorescence imaging is used to detect the bound complex and thereby determine the identity of the N+1 nucleotide in the target nucleic acid sequence (where the primer extension strand is N nucleotides in length). Following the imaging step, the multivalent binding complex is disrupted and washed away, the correct blocked nucleotide is incorporated into the primer extension strand, and the sequencing cycle is repeated

In some embodiments, nucleic acid hybridization is used for detecting the analytes. These methods utilize labeled nucleic acid probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), all of which are herein incorporated by reference in their entireties.

In some embodiments, a probe or probe set is a probe comprising a 3′ or 5′ overhang upon hybridization to the target nucleic acid. In some embodiments, the overhang comprises one or more barcode sequences corresponding to the target nucleic acid (e.g., the target RNA transcript). In some embodiments, a plurality of probes are designed to hybridize to the target nucleic acid (e.g., at least 20, 30, or 40 probes can hybridize to the target nucleic acid). In some embodiments, the probe or probe set is a probe comprising a 3′ overhang and a 5′ overhang upon hybridization to the target nucleic acid (a U-shaped probe). In some embodiments, the 3′ overhang and the 5′ overhang each independently comprises one or more detectable labels and/or barcode sequences. In some embodiments, the 3′ and/or 5′ overhang comprises one or more detectable labels and/or barcode sequences.

In some embodiments, analysis comprises using a codebook comprising signal code sequence that are sequences of color codes, arranged in the order of the corresponding signal color detected in sequential cycles of probe hybridization and imaging. In some aspects, the provided methods which immobilize and tether RNA in the biological sample are advantageous when using detection methods that comprise a plurality of repeated cycles of hybridization and removal of probes (e.g., detectably labeled probes, or intermediate probes that bind to detectably labeled probes) to the primary probe or probe set hybridized to the RNA, or to a rolling circle amplification product generated from the probe or probe set hybridized to the RNA.

### IV. Kits, Systems and Compositions

In some aspects, provided herein are subunits suitable for matrix formation, such as any of the multi-arm monomers or polymers or second monomers or polymers described herein.

In some embodiments, provided herein is a monomer or polymer of Formula (II-1)

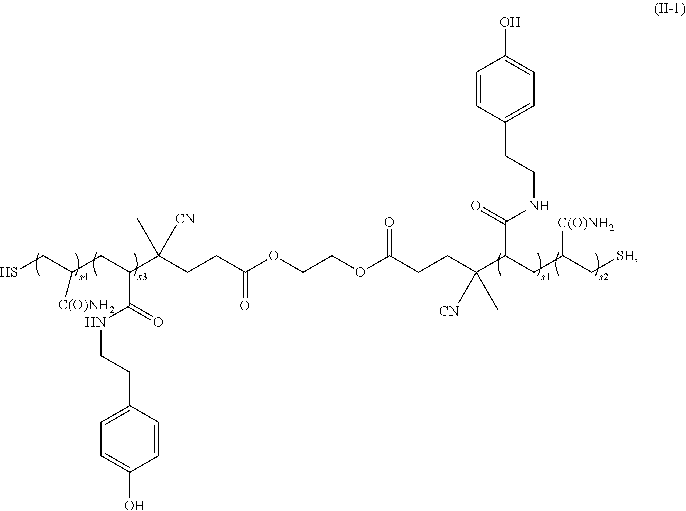

wherein ×1, ×2, ×3, and ×4 are each independently an integer from 1 to 10.

In some embodiments, provided herein is a monomer or polymer is of Formula (II-3)

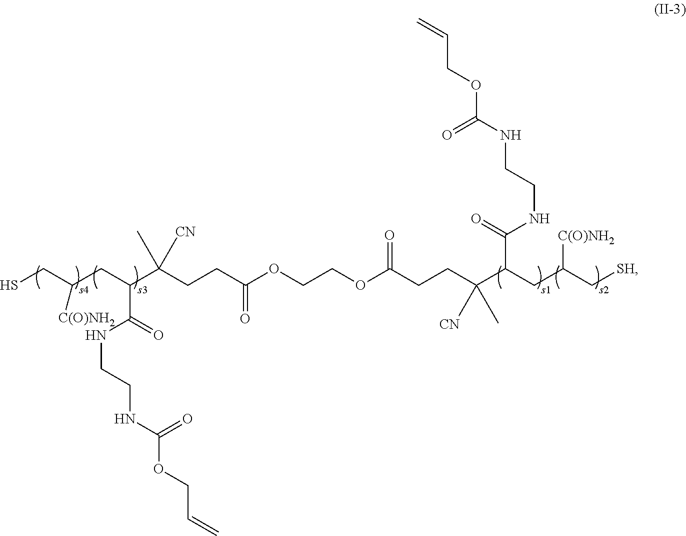

wherein s1, s2, s3, and s4 are each independently an integer from 1 to 10.

In some embodiments, provided herein is a monomer or polymer of Formula (I-1):

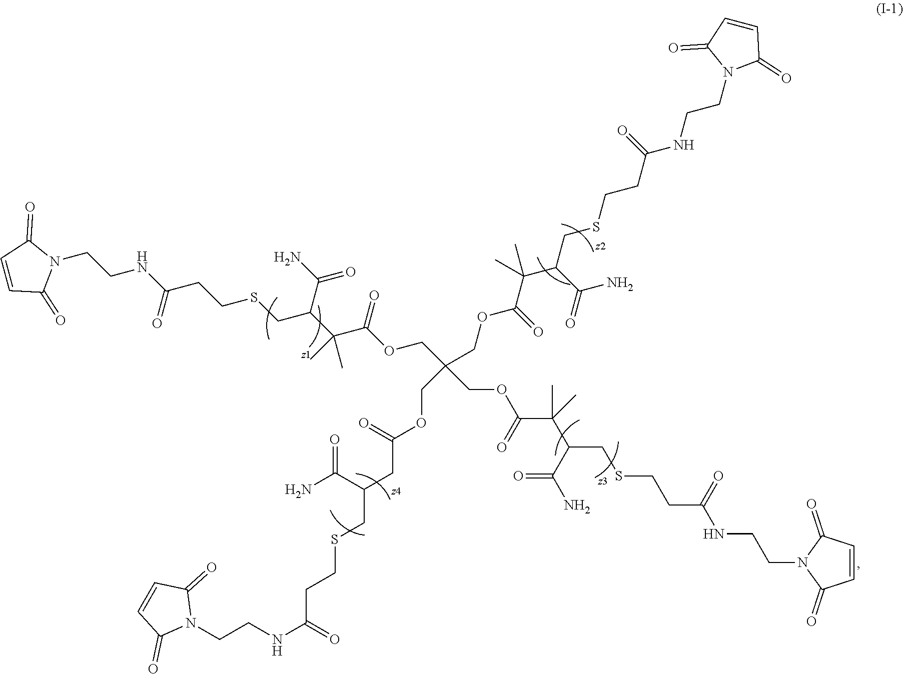

wherein z1, z2, z3, and z4 are each independently an integer from 1 to 10.

In some embodiments, provided herein is a monomer or polymer of Formula (II-2):

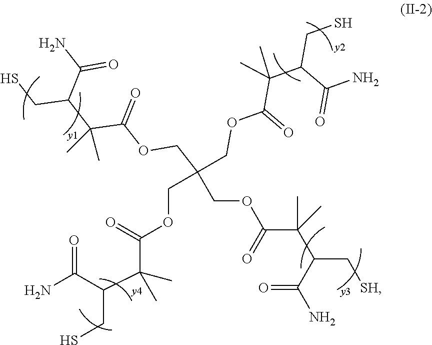

wherein y1, y2, y3, and y4 are each independently an integer from 1 to 10.

Also provided herein are matrices formed using any of the multi-arm monomers or polymers and second monomers or polymers described herein. In some embodiments, the matrix is a matrix embedding a biological sample. In one aspect, provided herein is a hydrogel matrix embedding a biological sample that is the product of a polymerization reaction between: (i) a multi-arm monomer or polymer comprising a plurality of arms converging at a central branching point, each arm comprising a functional group R^{A}, and (ii) a second monomer or polymer comprising at least two functional groups R^{B} and a pendant tethering moiety R^{T}, wherein the polymerization reaction is between R^{A} and R^{B}; and wherein R^{T} is attached to a cognate attachment moiety that is directly or indirectly attached to an analyte, a labeling agent associated with an analyte, or a nucleic acid amplification product, or any combination thereof in the biological sample.

Also provided herein are kits. In some aspects, provided herein is a kit for processing a biological sample (e.g., for embedding the biological sample in a matrix). In some embodiments, any one or more of the monomers or polymers described herein are premixed, stored, and/or shipped in one or more containers such as vials. In some embodiments, any one or more of the monomers or polymers described herein are provided separately. For instance, each of the multi-arm monomer or polymer and the second monomer or polymer can be provided, stored, and/or shipped in a separate container, although any two or more of the separate containers can be packaged together. In some embodiments, the monomer described herein are provided as a stock solution. In some embodiments, the kit comprises instructions for mixing a plurality of components together.

In one aspect, provided herein is a kit for processing a biological, comprising: (i) a multi-arm monomer or polymer comprising a plurality of arms converging at a central branching point, each arm comprising a functional group R^{A}, and (ii) a second monomer or polymer comprising at least two functional groups R^{B} and a pendant tethering moiety R^{T}, wherein R^{A} and R^{B} are capable of covalently reacting to crosslink the multi-arm monomer or polymer and the second monomer or polymer without reacting R^{T}; wherein R^{T} is capable of reacting with an attachment moiety using a chemistry orthogonal to the reaction between R^{A} and R^{B}.

In some embodiments, provided herein is a system comprising any of the kits described herein. In some embodiments, provided herein is a system comprising the kit and the biological sample. In some embodiments, provided herein is a system comprising any of the multi-arm monomers or polymers (e.g., as described in Section II) and one or more reagents for detecting an analyte, a labeling agent, or a nucleic acid amplification product in a biological sample (e.g., as described in Section III). In some embodiments, the system comprises reagents for performing RCA and/or reagents for detecting an RCA product. In some embodiments, the system comprises an opto-fluidic instrument (e.g., as described in Section V).

In some embodiments of the foregoing, the multi-arm monomer or polymer comprises any of the multi-arm monomers or polymers detailed herein. In some embodiments, the second monomer or polymer comprises any of the second monomers or polymers detailed herein.

The various components of the kit or system may be present in separate containers or certain compatible components may be pre-combined into a single container. In some embodiments, the kits further contain instructions for using the components of the kit to practice the provided methods.

In some embodiments, the kits or systems comprises reagents and/or consumables required for performing one or more steps of the provided methods. In some embodiments, the kits or systems contain reagents for fixing, embedding, and/or permeabilizing the biological sample. In some embodiments, the kits or systems contain reagents, such as enzymes and buffers for ligation and/or amplification, such as ligases and/or polymerases. In some aspects, provided is a kit or system that comprises any of the reagents described herein, e.g., any of the multi-arm monomers or polymers and/or any of the second monomers or polymers described herein. In some embodiments, provided is a kit or system that contain reagents for detection and/or sequencing, such as barcode detection probes or detectable labels. In some embodiments, the kits or systems optionally contain other components, for example nucleic acid primers, enzymes and reagents, buffers, nucleotides, modified nucleotides, reagents for additional assays. In some embodiments, the kits or systems comprises a substrate (e.g., a solid substrate configured to attach a cell or tissue sample).

### V. Opto-Fluidic Instruments for Analysis of Biological Samples

Provided herein is an instrument having integrated optics and fluidics modules (an “opto-fluidic instrument” or “opto-fluidic system”) for detecting target molecules (e.g., nucleic acids, proteins, antibodies, etc.) in biological samples (e.g., one or more cells or a tissue sample) as described herein. In an opto-fluidic instrument, the fluidics module is configured to deliver one or more reagents (e.g., detectably labeled probes) to the biological sample and/or remove spent reagents therefrom. Additionally, the optics module is configured to illuminate the biological sample with light having one or more spectral emission curves (over a range of wavelengths) and subsequently capture one or more images of emitted light signals from the biological sample during one or more probing cycles (e.g., as described in Section III-C). In some embodiments, tethering of analytes, probes, labeling agents or amplification products using attachment moieties described herein (e.g., as described in Section II) can help with cyclic assays where there are rounds of probe hybridization, unhybridization (e.g., by washing), and rehybridization, such that the assay (e.g., an in situ assay) can be performed using an automated instrument or system, e.g., an opto-fluidic instrument or system disclosed herein. In some embodiments, a biological sample is processed to tether of analytes, probes, labeling agents or amplification products using attachment moieties described herein (e.g., as described in Section II) prior to loading onto an automated instrument or system.

In various embodiments, the captured images may be processed in real time and/or at a later time to determine the presence of the one or more target molecules in the biological sample, as well as three-dimensional position information associated with each detected target molecule. Additionally, the opto-fluidics instrument includes a sample module configured to receive (and, optionally, secure) one or more biological samples. In some instances, the sample module includes an X-Y stage configured to move the biological sample along an X-Y plane (e.g., perpendicular to an objective lens of the optics module).

In various embodiments, the opto-fluidic instrument is configured to analyze one or more target molecules (e.g., any of the analytes described in Section III) in their naturally occurring place (i.e., in situ) within the biological sample. For example, an opto-fluidic instrument may be an in-situ analysis system used to analyze a biological sample and detect target molecules (e.g., analytes) including but not limited to DNA, RNA, proteins, antibodies, and/or the like.

It is to be noted that, although the above discussion relates to an opto-fluidic instrument that can be used for in situ target molecule detection via probe hybridization, the discussion herein equally applies to any opto-fluidic instrument that employs any imaging or target molecule detection technique. That is, for example, an opto-fluidic instrument may include a fluidics module that includes fluids needed for establishing the experimental conditions required for the probing of target molecules in the sample. Further, such an opto-fluidic instrument may also include a sample module configured to receive the sample, and an optics module including an imaging system for illuminating (e.g., exciting one or more fluorescent probes within the sample) and/or imaging light signals received from the probed sample. The in-situ analysis system may also include other ancillary modules configured to facilitate the operation of the opto-fluidic instrument, such as, but not limited to, cooling systems, motion calibration systems, etc.

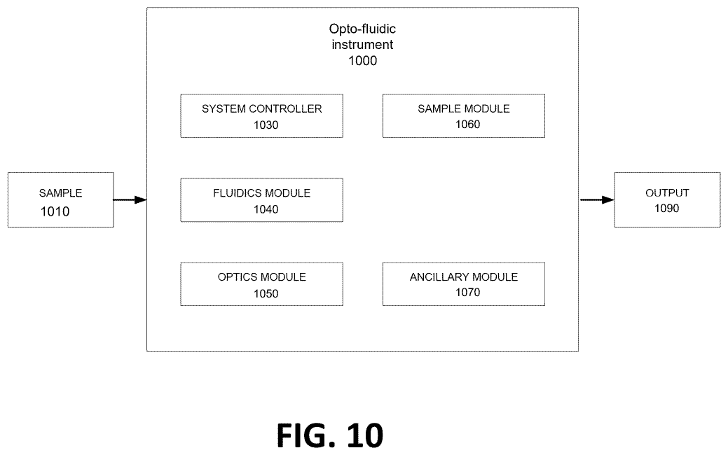

FIG. 10 shows an example workflow of analysis of a biological sample 1010 (e.g., cell or tissue sample) using an opto-fluidic instrument or system 1000, according to various embodiments. In various embodiments, the sample 1010 can be a biological sample (e.g., a tissue) that includes molecules such as DNA, RNA, proteins, antibodies, etc. For example, the sample 1010 can be a sectioned tissue that is treated to access the analytes for labeling with probes described herein (e.g., in Section III). Ligation of the probes may generate a circular probe which can be enzymatically amplified and bound with detectably labeled probes, which can create bright signal that is convenient to image and has a high signal-to-noise ratio.

In various embodiments, the sample 1010 (e.g., processed as described in Section II) is placed in the opto-fluidic instrument or system 1000 for analysis and detection of the molecules in the sample 1010. In various embodiments, the opto-fluidic instrument or system 1000 can be a system configured to facilitate the experimental conditions conducive for the detection of the target molecules. For example, the opto-fluidic instrument or system 1000 can include a fluidics module 1040, an optics module 1050, a sample module 1060, and an ancillary module 1070, and these modules may be operated by a system controller 1030 to create the experimental conditions for the probing of the molecules in the sample 1010 by selected probes or probe sets (e.g., circularizable DNA probes), as well as to facilitate the imaging of the probed sample (e.g., by an imaging system of the optics module 1050). In various embodiments, the various modules of the opto-fluidic instrument or system 1000 may be separate components in communication with each other, or at least some of them may be integrated together.

In various embodiments, the sample module 1060 may be configured to receive the sample 1010 into the opto-fluidic instrument or system 1000. For instance, the sample module 1060 may include a sample interface module (SIM) that is configured to receive a sample device (e.g., cassette) onto which the sample 1010 can be deposited. That is, the sample 1010 may be placed in the opto-fluidic instrument or system 1000 by depositing the sample 1010 (e.g., the sectioned tissue) on a sample device that is then inserted into the SIM of the sample module 1060. In some instances, the sample module 1060 may also include an X-Y stage onto which the SIM is mounted. The X-Y stage may be configured to move the SIM mounted thereon (e.g., and as such the sample device containing the sample 1010 inserted therein) in perpendicular directions along the two-dimensional (2D) plane of the opto-fluidic instrument or system 1000.

The experimental conditions that are conducive for the detection of the molecules in the sample 1010 may depend on the target molecule detection technique that is employed by the opto-fluidic instrument or system 1000. For example, in various embodiments, the opto-fluidic instrument or system 1000 can be a system that is configured to detect molecules in the sample 1010 via hybridization of probes. In such cases, the experimental conditions can include molecule hybridization conditions that result in the intensity of hybridization of the target molecule (e.g., nucleic acid) to a probe (e.g., oligonucleotide) being significantly higher when the probe sequence is complementary to the target molecule than when there is a single-base mismatch. The hybridization conditions include the preparation of the sample 1010 using reagents such as washing/stripping reagents, hybridizing reagents, etc., and such reagents may be provided by the fluidics module 1040.

In various embodiments, the fluidics module 1040 may include one or more components that may be used for storing the reagents, as well as for transporting said reagents to and from the sample device containing the sample 1010. For example, the fluidics module 1040 may include reservoirs configured to store the reagents, as well as a waste container configured for collecting the reagents (e.g., and other waste) after use by the opto-fluidic instrument or system 1000 to analyze and detect the molecules of the sample 1010. Further, the fluidics module 1040 may also include pumps, tubes, pipettes, etc., that are configured to facilitate the transport of the reagent to the sample device (e.g., and as such the sample 1010). For instance, the fluidics module 1040 may include pumps (“reagent pumps”) that are configured to pump washing/stripping reagents to the sample device for use in washing/stripping the sample 1010 (e.g., as well as other washing functions such as washing an objective lens of the imaging system of the optics module 1050).

In various embodiments, the ancillary module 1070 can be a cooling system of the opto-fluidic instrument or system 1000, and the cooling system may include a network of coolant-carrying tubes that are configured to transport coolants to various modules of the opto-fluidic instrument or system 1000 for regulating the temperatures thereof. In such cases, the fluidics module 1040 may include coolant reservoirs for storing the coolants and pumps (e.g., “coolant pumps”) for generating a pressure differential, thereby forcing the coolants to flow from the reservoirs to the various modules of the opto-fluidic instrument or system 1000 via the coolant-carrying tubes. In some instances, the fluidics module 1040 may include returning coolant reservoirs that may be configured to receive and store returning coolants, i.e., heated coolants flowing back into the returning coolant reservoirs after absorbing heat discharged by the various modules of the opto-fluidic instrument or system 1000. In such cases, the fluidics module 1040 may also include cooling fans that are configured to force air (e.g., cool and/or ambient air) into the returning coolant reservoirs to cool the heated coolants stored therein. In some instance, the fluidics module 1040 may also include cooling fans that are configured to force air directly into a component of the opto-fluidic instrument or system 1000 so as to cool said component. For example, the fluidics module 1040 may include cooling fans that are configured to direct cool or ambient air into the system controller 1030 to cool the same.

As discussed above, the opto-fluidic instrument or system 1000 may include an optics module 1050 which include the various optical components of the opto-fluidic instrument or system 1000, such as but not limited to a camera, an illumination module (e.g., LEDs), an objective lens, and/or the like. The optics module 1050 may include a fluorescence imaging system that is configured to image the fluorescence emitted by the probes (e.g., oligonucleotides) in the sample 1010 after the probes are excited by light from the illumination module of the optics module 1050.

In some instances, the optics module 1050 may also include an optical frame onto which the camera, the illumination module, and/or the X-Y stage of the sample module 1060 may be mounted.

In various embodiments, the system controller 1030 may be configured to control the operations of the opto-fluidic instrument or system 1000 (e.g., and the operations of one or more modules thereof). In some instances, the system controller 1030 may take various forms, including a processor, a single computer (or computer system), or multiple computers in communication with each other. In various embodiments, the system controller 1030 may be communicatively coupled with data storage, set of input devices, display system, or a combination thereof. In some cases, some or all of these components may be considered to be part of or otherwise integrated with the system controller 1030, may be separate components in communication with each other, or may be integrated together. In other examples, the system controller 1030 can be, or may be in communication with, a cloud computing platform.

In various embodiments, the opto-fluidic instrument or system 1000 may analyze the sample 1010 and may generate the output 1090 that includes indications of the presence of the target molecules in the sample 1010. For instance, with respect to the example embodiment discussed above where the opto-fluidic instrument or system 1000 employs a hybridization technique for detecting molecules, the opto-fluidic instrument or system 1000 may cause the sample 1010 to undergo successive rounds of detectably labeled probe hybridization (e.g., using two or more sets of fluorescent probes, where each set of fluorescent probes is excited by a different color channel) and be imaged to detect target molecules in the probed sample 1010. In such cases, the output 1090 may include optical signatures (e.g., a codeword) specific to each gene, which allow the identification of the target molecules.

### VI. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

A “primer” as used herein, in some embodiments, is an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

In some embodiments, “ligation” refers to the formation of a covalent bond or linkage between the termini of two or more nucleic acids, e.g., oligonucleotides and/or polynucleotides, in a template-driven reaction. The nature of the bond or linkage may vary widely and the ligation, in some embodiments, is carried out enzymatically or chemically. As used herein, ligations are usually carried out enzymatically to form a phosphodiester linkage between a 5′ carbon terminal nucleotide of one oligonucleotide with a 3′ carbon of another nucleotide.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following examples are included for illustrative purposes only and are not intended to limit the scope of the present disclosure.

### Example 1

This example describes a workflow for processing and analyzing a biological sample. The provided workflow includes embedding the biological sample in a matrix using orthogonal chemistries for matrix gelation and for covalent attachment of the matrix to an attachment moiety, which is directly or indirectly attached to an analyte or labeling agent associated with an analyte, or a nucleic acid amplification product in the biological sample, or any combination thereof.

First, as shown in FIG. 8, tissue sections are prepared and provided on a substrate according to standard methods for sample preparation. The substrate is an optically transparent substrate such as a glass slide or coverslip. As shown in the example workflow, examples of tissue preparation include formalin fixation (FF) and formalin-fixed, paraffin embedded (FFPE) tissues. In the case of FFPE tissue sections, the workflow may include a deparaffinization step.

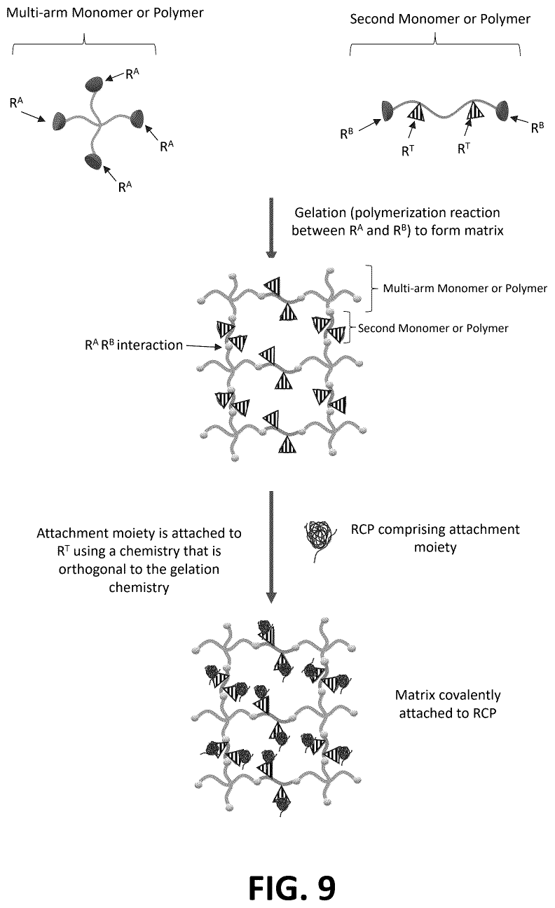

Next, the sample is embedded in a hydrogel matrix. This step comprises contacting the biological sample with: (i) a multi-arm monomer or polymer comprising a plurality of arms converging at a central branching point, each arm comprising a functional group R^{A}, and (ii) a second monomer or polymer comprising at least two functional groups R^{B} and a pendant tethering moiety R^{T}; and forming a matrix embedding the biological sample, wherein the matrix is the product of a polymerization reaction between the R^{A} of the multi-arm monomer or polymer and R^{B} of the second monomer or polymer. FIG. 9 provides a schematic illustration of the matrix formation using the multi-arm monomer or polymer and second monomer or polymer.

After matrix formation, the sample is permeabilized (FF tissue) or decrosslinked (FFPE tissue). Next, circularizable probes such as padlock probes are hybridized to target nucleic acids (e.g., mRNA molecules) in the matrix-embedded biological sample, and ligated using the target nucleic acids as templates to generate circularized probes. A 5′ acrydite modified primer is hybridized to the circularized probes. Rolling circle amplification is performed to generate RCA products using the circularized probes as templates. The RCA products are attached to R^{T} of the matrix using the 5′ acrydite attachment moiety (e.g., as illustrated in FIG. 9). In this example, R^{T} is acrylamide, which reacts with the 5′ acrydite to covalently attach the RCA products to the matrix.

In this example, R^{T} is covalently attached to the attachment moiety using acrylamide coupling. In an example, the orthogonal gelation chemistry for attachment of R^{A} to R^{B} is selected from the group consisting of Horseradish peroxidase (HRP) catalyzed coupling, Michael addition, Diels Alder coupling (e.g., maleimide/furan coupling) ally-thiol coupling, click reaction (e.g., alkyne/azide), Tyrosinase-catalyzed coupling (e.g., coupling of phenol-amine), Transglutaminase-catalyzed coupling (e.g., coupling of thiol-maleimide), Cu-catalyzed click coupling, light-catalyzed coupling (e.g., sulfur/allyl ester coupling and sulfide/norbornene coupling).

In an example, the orthogonal gelation chemistry uses thiol-maleimide coupling, Examples of thiol-maleimide coupling matrix-forming agents including the multi-arm monomer or polymer and the second monomer or polymer are provided in FIG. 2A, FIG. 2B, FIG. 2C, and FIG. 5, and are described throughout the present disclosure.

In the example workflow provided in FIG. 8, the method comprises performing tissue clearing after performing the orthogonal crosslinking chemistry to attach the RCPs to the matrix. The tissue clearing reduces autofluorescence by removing sources of autofluorescence in the biological sample (e.g., proteins and/or lipids).

After tissue clearing, multiple cycles of probe hybridization or sequencing are performed to analyze sequences of the RCPs (e.g., barcode sequences). The RCPs remain tethered to the matrix, providing improved positional stability for multiple cycles of probe hybridization or sequencing.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A method for processing a biological sample, the method comprising:
a) contacting the biological sample with: (i) a multi-arm monomer or polymer comprising a plurality of arms converging at a central branching point, each arm comprising a functional group R^{A}, and (ii) a second monomer or polymer comprising at least two functional groups R^{B} and a pendant tethering moiety R^{T};
b) forming a matrix embedding the biological sample, wherein the matrix is the product of a polymerization reaction between the R^{A} of the multi-arm monomer or polymer and R^{B} of the second monomer or polymer; and
c) attaching R^{T} to an attachment moiety that is directly or indirectly attached to an analyte or labeling agent associated with an analyte, or a nucleic acid amplification product in the biological sample, or any combination thereof;
thereby attaching the analyte, labeling agent, or nucleic acid amplification product, or any combination thereof to the matrix.

2-15. (canceled)

16. The method of claim 1, wherein the polymerization reaction and the attaching of R^{T} to the attachment moiety are performed using orthogonal chemical reactions.

17-21. (canceled)

22. The method of claim 1, wherein:
(i) the multi-arm monomer or polymer is a compound according to Formula I: 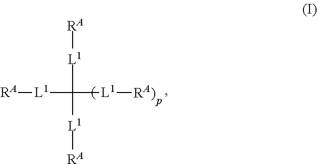
or a salt thereof,
wherein p is 0 or 1;
wherein L¹ is an C₅-C₁₀₀ alkylene optionally interrupted by one or more groups independent selected from the group consisting of —NH—, —O—, —S—, —SO₂—, —N(C₁₋₆ alkyl)-, C₆-C₁₀ aryl, 5- to 6-membered heteroaryl, C₃-C₈ cycloalkyl, and 5- to 6-membered heterocycle, and L′ is optionally substituted with one or more substituents independently selected from the group consisting of oxo, halo, —OH, —CN, C₁-C₆ alkoxy, C₁-C₆ haloalkoxy, or a side chain of an amino acid;
and
(ii) the second monomer or polymer is a compound according to Formula II: 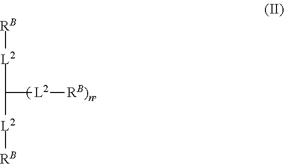
or a salt thereof,
wherein n is 0, 1, or 2;
wherein L² is an C₅-C₁₀₀ alkylene optionally interrupted by one or more groups independent selected from the group consisting of —NH—, —O—, —S—, —SO₂—, —N(C₁₋₆ alkyl)-, a C₆-C₁₀ aryl, 5- to 6-membered heteroaryl, C₃-C₈ cycloalkyl, and 5- to 6-membered heterocycle, and L² is optionally substituted with one or more substituents independently selected from the group consisting of oxo, halo, —OH, —CN, amide, C₁-C₆ alkoxy, C₁-C₆ haloalkoxy, and a side chain of an amino acid;
wherein L² is further attached to one or more tethering moiety R^{T};
wherein R^{A} is capable of reacting with R^{B} to form the matrix of b); and
wherein R^{T} is substantially unreacted when R^{A} reacts with R^{B} to form the matrix in b).

23. (canceled)

24. The method of claim 22, wherein L¹ comprises a PEG unit or a polyacrylamide unit and L² comprises a PEG unit or a polyacrylamide unit.

25-28. (canceled)

29. The method of claim 1, wherein R^{A} is a thiol group and R^{B} is a maleimide group, or wherein R^{A} is a maleimide group and R^{B} is a thiol group.

30-32. (canceled)

33. The method of claim 1, wherein the second monomer or polymer is of Formula (II-1) 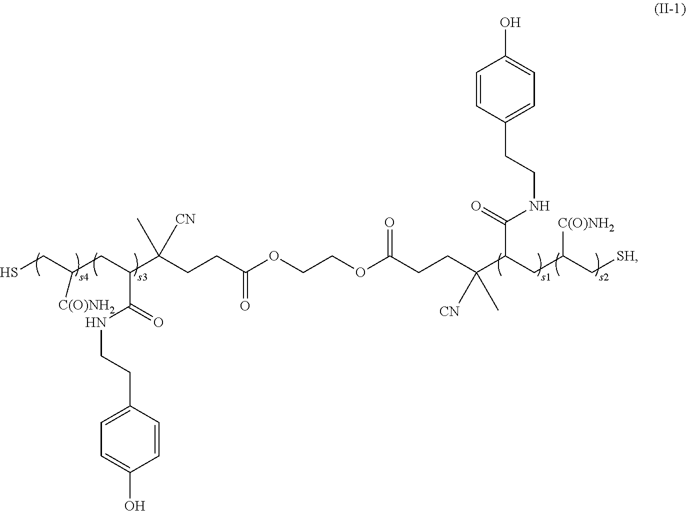
wherein ×1, ×2, ×3, and ×4 are each independently an integer from 1 to 10.

34. (canceled)

35. The method of claim 1, wherein the second monomer or polymer is of Formula (II-2): 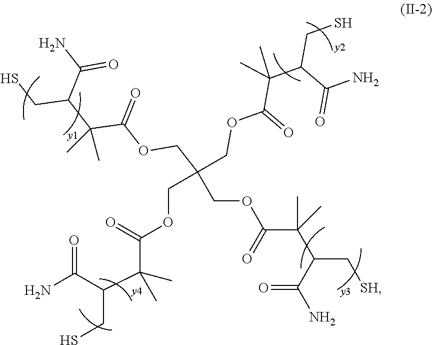
wherein y1, y2, y3, and y4 are each independently an integer from 1 to 10.

36. (canceled)

37. The method of claim 1, wherein the multi-arm monomer or polymer is of Formula (I-1) 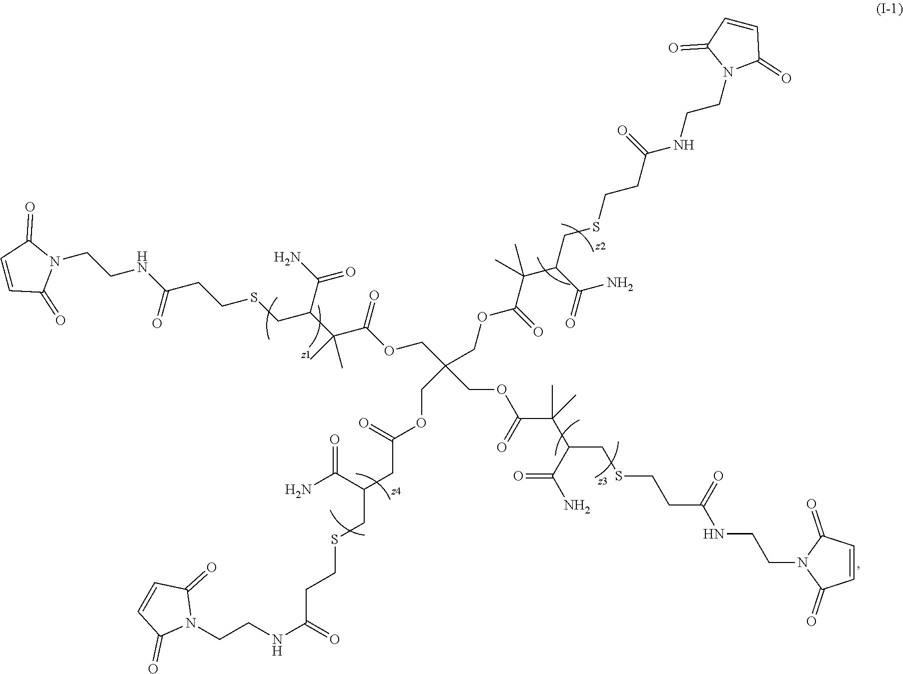
wherein 21, 22, 23, and z4 are each independently an integer from 1 to 10.

38-42. (canceled)

43. The method of claim 1, wherein (i) R^{T} comprises a phenol group and the attachment moiety comprises an amine group, or (ii) R^{T} comprises an amine group and the attachment moiety comprises a phenol group,
wherein attaching R^{T} to the attachment moiety comprises performing a tyrosinase reaction; or wherein (i) R^{T} comprises an alkene group and the attachment moiety comprises a thiol group, or (ii) R^{T} comprises a thiol group and the attachment moiety comprises an alkene group, wherein attaching R^{T} to the attachment moiety comprises performing a thiol-ene addition in the presence of ultraviolet irradiation.

44-46. (canceled)

47. The method of claim 1, wherein the biological sample is on a chemically modified substrate, wherein the chemically modified substrate is capable of immobilizing the multi-arm monomer or polymer, the second monomer or polymer, or both.

48. (canceled)

49. The method of claim 47, wherein the chemically modified substrate is capable of forming covalent bond with R^{A} of the multi-arm monomer or polymer, R^{B} of the second monomer or polymer, or R^{T} of the second monomer or polymer.

50. The method of claim 47, wherein the chemically modified substrate comprises an NHS ester, and at least one of R^{A}, R^{B}, or R^{T} comprises —NH₂.

51. (canceled)

52. The method of claim 47, wherein the chemically modified substrate is capable of forming covalent bond with a linking reagent, and the linking reagent is capable of forming covalent bond with R^{A} of the multi-arm monomer or polymer, R^{B} of the second monomer or polymer, or R^{T} of the second monomer or polymer.

53. (canceled)

54. (canceled)

55. The method of claim 1, wherein the analyte is a nucleic acid analyte, optionally wherein the analyte is an RNA present in the biological sample.

56. The method of claim 55, wherein the method comprises contacting the biological sample or the matrix with a nucleic acid probe or probe set that hybridizes to a target sequence in the nucleic acid analyte, wherein the attachment agent is directly or indirectly attached to a labeling agent that binds to the nucleic acid analyte, and wherein the labeling agent is the nucleic acid probe or probe set.

57. (canceled)

58. The method of claim 56, wherein the nucleic acid probe or probe set is (i) a circular probe, or (ii) a circularizable probe or probe set, wherein the method comprises circularizing the circularizable probe or probe set to form a circularized probe.

59. The method of claim 58, wherein the method comprises generating a rolling circle amplification (RCA) product from the circular or circularized probe in the biological sample or matrix.

60. (canceled)

61. The method of claim 59, wherein the attachment moiety is in a primer used to form the RCA product, and wherein the primer comprises a reverse complement of a primer binding sequence in the circular or circularized probe.

62. The method of claim 59, wherein the attachment moiety is in a modified nucleotide that is incorporated into the RCA product.

63-68. (canceled)

69. The method of claim 1, wherein the method comprises analyzing a nucleotide sequence of the analyte, labeling agent, or nucleic acid amplification product at a location in the biological sample or the matrix.

70. The method of claim 69, wherein the nucleotide sequence is analyzed by sequential hybridization, sequencing by hybridization, sequencing by ligation, sequencing by synthesis, sequencing by binding, or a combination thereof.

71. (canceled)

72. (canceled)

73. The method of claim 1, wherein the biological sample is a tissue sample.

74-86. (canceled)
